SEQUENCE LISTING (28 sequences)

SEQ ID NO: 1 - DNA, 2065 bp, Homo sapien (human)
attcgggaccttggggcagctcctgagttcagacagagttcaggaagggagacaggggcacagagagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcagcggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtgatcctgctggaagaggcgtggagtgaactctttctcctcggggccatccagtggtctctgcctctggacagctgtcctctgctggcaccgcccgaggcttctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcaaggcccaccaccccagccagcccgtgaggtgacctgagcatgcgcccacccactcatctgtccctgacctctaacctttctctgcctctcccacactctcccagagctcactgattagacagcacaagggtctcagttcaacagcatacagccaacatctatggtgtcccaggcacagtgccaggccccgggagtggggaccaagatgtacataagacaaagctactgccttctagagacaaccggcagtgacctcactgaagacaaaaactgccctagccaggtactgagggttgcatgaatctgcaggagacagagatccccttgcatgggaaacataaagcagaattgggagggactttgtggagacagggctggacttgaaaggaagaagaagtctaaaagaaaacatcatttgcaaagggagagaggggcaagcatgatatgttgttagaacaggagcccactttgaaggtataacaggttcctgccagtgagaaatggggagaataagccagaaaagtaccctaggaccagcccgttcaggactttgaatgccagccaaaggccacgtctgacttgggaggcagagggcagctactgcaggtttccgagcagagggtcatacacagggctggacctcacgcagactggcatggccatgggtccagaggatactactgggaaggggatggcagctactgccaccttccagatggttccatggagttctgatctttgggcatggccaggggaagcagaagggagactctaggagttgaaatgggtcagacccggtgtttgggtgaaggtaaggaatgagggaagaggagctctttg

SEQ ID NO: 2 - protein, 367 aa, Homo sapien (human)
METRPTALMSSTVAAAAPAAGAASRKESPGRWGLGEDPTGVSPSLQCRVCGDSSSGKHYGIYACNGCSGFFKRSVRRRLIYRCQVGAGMCPVDKAHRNQCQACRLKKCLQAGMNQDAVQNERQPRSTAQVHLDSMESNTESRPESLVAPPAPAGRSPRGPTPMSAARALGHHFMASLITAETCAKLEPEDADENIDVTSNDPEFPSSPYSSSSPCGLDSIHETSARLLFMAVKWAKNLPVFSSLPFRDQVILLEEAWSELFLLGAIQWSLPLDSCPLLAPPEASAAGGAQGRLTLASMETRVLQETISRFRALAVDPTEFACMKALVLFKPETRGLKDPEHVEALQDQSQVMLSQHSKAHHPSQPVR

SEQ ID NO: 3 - DNA, 860 bp, Homo sapien (human)
  misc_feature (22)...(860): n = A, T, C or G
ggaatcaccaggggagacaggngcacagngagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgccctgcaacggttgcagcggtttcttccaagaggagcngtacggnggaggctcaatccttacaagggtgcccagggtgggggcagggattgtgccccccngtggacaaggncccaacccgnaacccagtgcccaggcctgccggnttgagaagtgcttnaaaannnggnnggggnttgaacccaggacgcccgtnnaaaggaacganngccnagcccgngagganaagcccaggtnccacccctgganaagaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 4 - DNA, 20 bp, Artificial Sequence
  Oligonucleotide
atgagctccacagtggctgc

SEQ ID NO: 5 - DNA, 20 bp, Artificial Sequence
  Oligonucleotide
ctgtctccgcacacgcggca

SEQ ID NO: 6 - DNA, 20 bp, Artificial Sequence
  Oligonucleotide
tttcgagcttccaggttcat

SEQ ID NO: 7 - DNA, 20 bp, Artificial Sequence
  Oligonucleotide
ctcccaaactctgcctggtg

SEQ ID NO: 8 - DNA, 20 bp, Artificial Sequence
  Oligonucleotide
cgggagccacacttcaccat

SEQ ID NO: 9 - DNA, 20 bp, Artificial Sequence
  Oligonucleotide
gctcacttctgcgctgtctg

SEQ ID NO: 10 - DNA, 20 bp, Artificial Sequence
  Oligonucleotide
ttccgggctcccagagtcat

SEQ ID NO: 11 - DNA, 20 bp, Artificial Sequence
  Oligonucleotide
cagaagacctgcctgatctg

SEQ ID NO: 12 - DNA, 20 bp, Artificial Sequence
  Oligonucleotide
gaaatgaactccttcatcat

SEQ ID NO: 13 - DNA, 20 bp, Artificial Sequence
  Oligonucleotide
ccggatctgtggggtgtgtg

SEQ ID NO: 14 - DNA, 19 bp, Artificial Sequence
  Oligonucleotide
ctgatgagaatattgatgt

SEQ ID NO: 15 - DNA, 19 bp, Artificial Sequence
  Oligonucleotide
cgtgagccggccctgggca

SEQ ID NO: 16 - DNA, 20 bp, Artificial Sequence
  Oligonucleotide
ggcatggacctcactgaaga

SEQ ID NO: 17 - DNA, 20 bp, Artificial Sequence
  Oligonucleotide
actggcaggaacctgttata

SEQ ID NO: 18 - DNA, 3012 bp, Homo sapien (human)
tatagggcgaattgggtaccgggccccccctcgaggtcgacggtatcgataagcttgatatcgaattcgaattcgggaccttggggcagctcctgagttcagacagagttcaggaagggagacaggggcacagagagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcagcggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtacctaccggcctgcctgctggggagctaggctgggctggggtcaggcggcccactcgagtcaaccagacagggcacacacatccccacgccagtatgaatgcacacagcttggatggtgatggctggggacacacatacctctgattcagcgatggctggggtgcatctcagggatggtgacggtgggggtgcatgcatctctggcacagggatgatggtcggggtgcacacctaggagatgatgatggctagggacctacagggcccagggtcttcttaagttctggaagaccctcaggccctgcagacattctgtgggtaacaagtgacctgcacaccctgaacaggctgagtggctgactctaggcccccttggagcacaagtgcctacgacttcagggcttgcattttagttcaatctctccagctctgggccatccctctcggcttctaatgggcaagcagatctttcaggaaaaccaggaggagaggcatgaggaaggtttgaggccctcagccagtctgtgtgctggggtggagcaactcagaagagtcaggccacaccacttgaatacactcaacttaggacactcatgaggcatgtctctgaggctgcccaacttccaatggctctgggcgttcctaaatgtcccagctgcagctctggatggaacccagtgtctcagatgataggcagctgagccggatggtgccaaatcccagagctctgagcctctggctgatgtcaggagagcattctcgggtcccaggacagcacttccattccttgggtgcctgagatggtggcagaggctccagactgagccagagaagctgtgtgtctgccataacaggcacccctgtctgagcacaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcaaggcccaccaccccagccagcccgtgaggtgacctgagcatgcgcccacccactcatctgtccctgacctctaacctttctctgcctctcccacactctcccagagctcactgattagacagcacaagggtctcagttcaacagcatacagccaacatctatggtgtcccaggcacagtgccaggccccgggagtggggaccaagatgtacataagacaaagctactgccttctagagacaaccggcagtgacctcactgaagacaaaaactgccctagccaggtactgagggttgcatgaatctgcaggagacagagatccccttgcatgggaaacataaagcagaattgggagggactttgtggagacagggctggacttgaaaggaagaagaagtctaaaagaaaacatcatttgcaaagggagagaggggcaagcatgatatgttgttagaacaggagcccactttgaaggtataacaggttcctgccagtgagaaatggggagaataagccagaaaagtaccctaggaccagcccgttcaggactttgaatgccagccaaaggccacgtctgacttgggaggcagagggcagctactgcaggtttccgagcagagggtcatacacagggctggacctcacgcagactggcatggccatgggtccagaggatactactgggaaggggatggcagctactgccaccttccagatggttccatggagttctgatctttgggcatggccaggggaagcagaagggagactctaggagttgaaatgggtcagacccggtgtttgggtgaaggtaaggaatgagggaagaggagctctttg

SEQ ID NO: 19 - DNA, 2135 bp, Homo sapien (human)
tatagggcgaattgggtaccgggccccccctcgaggtcgacggtatcgataagcttgatatcgaattcgaattcgggaccttggggcagctcctgagttcagacagagttcaggaagggagacaggggcacagagagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcagcggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcaaggcccaccaccccagccagcccgtgaggtgacctgagcatgcgcccacccactcatctgtccctgacctctaacctttctctgcctctcccacactctcccagagctcactgattagacagcacaagggtctcagttcaacagcatacagccaacatctatggtgtcccaggcacagtgccaggccccgggagtggggaccaagatgtacataagacaaagctactgccttctagagacaaccggcagtgacctcactgaagacaaaaactgccctagccaggtactgagggttgcatgaatctgcaggagacagagatccccttgcatgggaaacataaagcagaattgggagggactttgtggagacagggctggacttgaaaggaagaagaagtctaaaagaaaacatcatttgcaaagggagagaggggcaagcatgatatgttgttagaacaggagcccactttgaaggtataacaggttcctgccagtgagaaatggggagaataagccagaaaagtaccctaggaccagcccgttcaggactttgaatgccagccaaaggccacgtctgacttgggaggcagagggcagctactgcaggtttccgagcagagggtcatacacagggctggacctcacgcagactggcatggccatgggtccagaggatactactgggaaggggatggcagctactgccaccttccagatggttccatggagttctgatctttgggcatggccaggggaagcagaagggagactctaggagttgaaatgggtcagacccggtgtttgggtgaaggtaaggaatgagggaagaggagctctttg

SEQ ID NO: 20 - DNA, 1623 bp, Mu musculus (mouse)
tcggttgggcccagcaacttctagcaagcaggctacccttaggaccatccatatccgatgagctctacagtggctgcctccactatgcctgtgtctgtggcggcctccaagaaggagtctccaggtagatggggccttggagaggatccaacaggtgtgggcccctcgctccagtgccgagtgtgtggggacagcagcagtgggaaacattatggcatctatgcctgcaatggctgcagtggcttcttcaagaggagtgtgagaaggaggctcatctacaggtgccaagtcggggcagggatgtgcccagtggataaggcccatcgcaatcagtgccaggcctgccggctgaagaagtgcttacaagcaggcatgaaccaagatgctgtgcagaatgagcgccaacctcggagcatggctcaggtccacctggatgccatggaaacaggcagtgacccccgatcagaaccagtggtagcctctcctgctctggcagggcccagtccccggggccccacgtctgtgtctgcaaccagagccatgggccaccactttatggccagccttatcaccgccgaaacttgtgctaaactggagccagaggacgctgaagagaatattgatgtcaccagcaatgaccccgagttccccgcatccccctgcagtctggatggcatccatgagacatctgctcgcctgctcttcatggctgtcaaatgggccaaaaacttgcctgtgttttccaacctgcctttccgggaccaggtgatcttgctggaagaggcatggaatgagcttttccttcttggagccatacagtggtctctgcccctggacagctgcccactgctggcaccacctgaggcgtccggcagctctcagggcaggctggccttggccagtgcagagacgcgcttcctgcaggaaaccatctcccggttccgagctctggcagtggatcccacagagtttgcctgcctgaaggccctggtcctcttcaaacctgaaacacgaggcctgaaggatcctgagcacgtggaggctttgcaggaccagtcccaggtgatgctaagccagcatagcaaggctcaccaccccagccagcctgtgaggtttgggaaattgctcctcctgctcccatctttgaggttcctcacggctgagcgcattgagcttctcttcttcagaaagaccatagggaacactccgatggagaagctcctgtgtgacatgttcaaaaactagttgggagtgccaagtgtccacaggcacccaggggggcagcacatcttagaagctaaatagttccctgcctttctcagccagtaattccacattcaggtattcctacctagcagaaatttctcccaaaatatattattggcatattcattgccatcctaatcttaatacccctaactctgcttgggcagtagaatgcatggatgcgttgttatattcataggagaaacagctttggcaaaaaaaaaaaaaaaaaaaaaaaaaaaaaaaaaaaaaaaaaaaaaaaaactcgagggggggcccggtacccaattcgccctatagtgagtcgtattacaattcactggccgtcgttttacaacgtcgtgactgggaaaaccc

SEQ ID NO: 21 - protein, 395 aa, Mus musculus (mouse)
MSSTVAASTMPVSVAASKKESPGRWGLGEDPTGVGPSLQCRVCGDSSSGKHYGIYACNGCSGFFKRSVRRRLIYRCQVGAGMCPVDKAHRNQCQACRLKKCLQAGMNQDAVQNERQPRSMAQVHLDAMETGSDPRSEPVVASPALAGPSPRGPTSVSATRAMGHHFMASLITAETCAKLEPEDAEENIDVTSNDPEFPASPCSLDGIHETSARLLFMAVKWAKNLPVFSNLPFRDQVILLEEAWNELFLLGAIQWSLPLDSCPLLAPPEASGSSQGRLALASAETRFLQETISRFRALAVDPTEFACLKALVLFKPETRGLKDPEHVEALQDQSQVMLSQHSKAHHPSQPVRFGKLLLLLPSLRFLTAERIELLFFRKTIGNTPMEKLLCDMFKN

SEQ ID NO: 22 - DNA, 1623 bp, Mus musculus (mouse)
agccaacccgggtcgttgaagatcgttcgtccgatgggaatcctggtaggtataggctactcgagatgtcaccgacggaggtgatacggacacagacaccgccggaggttcttcctcagaggtccatctaccccggaacctctcctaggttgtccacacccggggagcgaggtcacggctcacacacccctgtcgtcgtcaccctttgtaataccgtagatacggacgttaccgacgtcaccgaagaagttctcctcacactcttcctccgagtagatgtccacggttcagccccgtccctacacgggtcacctattccgggtagcgttagtcacggtccggacggccgacttcttcacgaatgttcgtccgtacttggttctacgacacgtcttactcgcggttggagcctcgtaccgagtccaggtggacctacggtacctttgtccgtcactgggggctagtcttggtcaccatcggagaggacgagaccgtcccgggtcaggggccccggggtgcagacacagacgttggtctcggtacccggtggtgaaataccggtcggaatagtggcggctttgaacacgatttgacctcggtctcctgcgacttctcttataactacagtggtcgttactggggctcaaggggcgtagggggacgtcagacctaccgtaggtactctgtagacgagcggacgagaagtaccgacagtttacccggtttttgaacggacacaaaaggttggacggaaaggccctggtccactagaacgaccttctccgtaccttactcgaaaaggaagaacctcggtatgtcaccagagacggggacctgtcgacgggtgacgaccgtggtggactccgcaggccgtcgagagtcccgtccgaccggaaccggtcacgtctctgcgcgaaggacgtcctttggtagagggccaaggctcgagaccgtcacctagggtgtctcaaacggacggacttccgggaccaggagaagtttggactttgtgctccggacttcctaggactcgtgcacctccgaaacgtcctggtcagggtccactacgattcggtcgtatcgttccgagtggtggggtcggtcggacactccaaaccctttaacgaggaggacgagggtagaaactccaaggagtgccgactcgcgtaactcgaagagaagaagtctttctggtatcccttgtgaggctacctcttcgaggacacactgtacaagtttttgatcaaccctcacggttcacaggtgtccgtgggtccccccgtcgtgtagaatcttcgatttatcaagggacggaaagagtcggtcattaaggtgtaagtccataaggatggatcgtctttaaagagggttttatataataaccgtataagtaacggtaggattagaattatggggattgagacgaacccgtcatcttacgtacctacgcaacaatataagtatcctctttgtcgaaaccgtttttttttttttttttttttttttttttttttttttttttttttttttgagctcccccccgggccatgggttaagcgggatatcactcagcataatgttaagtgaccggcagcaaaatgttgcagcactgacccttttggg

SEQ ID NO: 23 - DNA, 25 bp, Artificial Sequence
  Oligonucleotide
cccaggctttacactttatgcttcc

SEQ ID NO: 24 - DNA, 20 bp, Artificial Sequence
  Oligonucleotide
tgccgcgtgtgcggagacag

SEQ ID NO: 25 - DNA, 21 bp, Artificial Sequence
  Oligonucleotide
gtaccgagctcggatccacta

SEQ ID NO: 26 - DNA, 21 bp, Artificial Sequence
  Oligonucleotide
ccgccagtgtgatggatatct

SEQ ID NO: 27 - DNA, 21 bp, Artificial Sequence
  Oligonucleotide
tcggttgggcccagcaacttc

SEQ ID NO: 28 - DNA, 20 bp, Artificial Sequence
  Oligonucleotide
ggggatgtgctgcaaggcga

(19) United States
(12) Patent Application Publication
(10) Pub. No.: US 2003/0096348 A1
(43) Pub. Date: May 22, 2003

(54) DNA molecules encoding mammalian nuclear receptor protein, nNR5

(75) Inventor: Fang Chen, North Wales, PA (US)

Correspondence Address: MERCK AND CO INC, P O BOX 2000, RAHWAY, NJ, 070650907

(73) Assignee: Merck & Co., Inc., Rahway, NJ

(21) Appl. No.: 10/151,542

(22) Filed: May 20, 2002

Related U.S. Application Data
(63) Continuation of application No. 09/326,755, filed on Jun. 4, 1999, now abandoned.
Application No. 09/326,755 is a continuation-in-part of application No. PCT/US98/26422, filed on Dec. 11, 1998.
(60) Provisional application No. 60/069,379, filed on Dec. 12, 1997.

(51) Int. Cl. C12P 21/02; C12N 5/06; C07K 14/47; C07H 21/04

(52) U.S. Cl. 435/69.1; 435/320.1; 435/325; 530/350; 536/23.5

(57) ABSTRACT

The present invention discloses the isolation and characterization of cDNA molecules encoding novel members to the human and mouse nuclear receptor superfamiliy, designated nNR5. Also within the scope of the disclosure are recombinant vectors, recombinant host cells, methods of screening for modulators of human and mouse nNR5 activity, production of antibodies against human and mouse nNR5, or epitopes thereof, as well as non-human animal modified to study the biological effect of various forms of nNR5.

### CROSS-REFERENCE TO RELATED APPLICATIONS

This application is a continuation-in-part application of PCT International Application No. PCT/US98/26422, filed Dec. 11, 1998, which is entitled to priority to U.S. Provisional application No. 60/069,379 filed Dec. 12, 1997.

### STATEMENT REGARDING FEDERALLY-SPONSORED R&D

Not applicable.

### REFERENCE TO MICROFICHE APPENDIX

Not applicable.

### FIELD OF THE INVENTION

The present invention relates in part to isolated nucleic acid molecules (polynucleotides) which encode vertebrate nuclear receptor proteins, and especially human and mouse nuclear receptor proteins as exemplified throughout this specification as human nNR5 and mouse nNR5. The present invention also relates to recombinant vectors and recombinant hosts which contain a DNA fragment encoding nNR5, substantially purified forms of associated human and mouse nNR5 protein, human and mouse mutant proteins, methods associated with identifying compounds which modulate nNR5 activity, and non-human animals which have been subject to intervention to effect nNR5 activity.

### BACKGROUND OF THE INVENTION

The nuclear receptor superfamily, which includes steroid hormone receptors, are small chemical ligand-inducible transcription factors which have been shown to play roles in controlling development, differentiation and physiological function. Isolation of cDNA clones encoding nuclear receptors reveal several characteristics. First, the NH₂-terminal regions, which vary in length between receptors, is hypervariable with low homology between family members. There are three internal regions of conservation, referred to as domain I, II and III. Region I is a cysteine-rich region which is referred to as the DNA binding domain (DBD). Regions II and III are within the COOH-terminal region of the protein and is also referred to as the ligand binding domain (LBD). For a review, see Power et al. (1992, Trends in Pharmaceutical Sciences 13: 318-323).

The lipophilic hormones that activate steroid receptors are known to be associated with human diseases. Therefore, the respective nuclear receptors have been identified as possible targets for therapeutic intervention. For a review of the mechanism of action of various steroid hormone receptors, see Tsai and O'Malley (1994, Annu. Rev. Biochem. 63: 451-486).

Recent work with non-steroid nuclear receptors has also shown the potential as drug targets for therapeutic intervention. This work reports that peroxisome proliferator activated receptor g (PPARg), identified by a conserved DBD region, promotes adipocyte differentiation upon activation and that thiazolidinediones, a class of antidiabetic drugs, function through PPARg (Tontonoz et al., 1994, Cell 79: 1147-1156; Lehmann et al., 1995, J. Biol. Chem. 270(22): 12953-12956; Teboul et al., 1995, J. Biol. Chem. 270(47): 28183-28187). This indicates that PPARg plays a role in glucose homeostasis and lipid metabolism.

Kobayashi et al. (Proc. Natl. Acad. Sci 96: 4814-4819) describe a cDNA which encodes a human retinal specific nuclear receptor which is a splice variant of the human nNR5 cDNA clone disclosed herein.

Wang et al. (1989, Nature 340: 163-166) show data which prompted the authors to classify the COUP transcription factor (COUP-TF) as a member of the nuclear receptor superfamily.

Mangelsdorf et al. (1995, Cell 83: 835-839) provide a review of known members of the nuclear receptor superfamily.

It would be advantageous to identify additional genes which are members of the nuclear receptor superfamily, especially vertebrate members from such species as human, rat and mouse. A nucleic acid molecule expressing a nuclear receptor protein will be useful in screening for compounds acting as a modulator of cell differentiation, cell development and physiological function. The present invention addresses and meets these needs by disclosing isolated nucleic acid molecules which express a human or mouse nuclear receptor protein which will have a role in cell differentiation and development.

### SUMMARY OF THE INVENTION

The present invention relates to isolated nucleic acid molecules (polynucleotides) which encode novel nuclear receptor proteins which are herein designated as members of the nuclear receptor superfamily. The isolated polynucleotides of the present invention encode vertebrate members of this nuclear receptor superfamily, and preferably human or mouse nuclear receptor proteins, such as the human and mouse nuclear receptor proteins exemplified and referred to throughout this specification as nNR5. The nuclear receptor proteins encoded by the isolated polynucleotides of the present invention are involved in the regulation of in vivo cell proliferation and/or cell development.

The present invention also relates to isolated nucleic acid fragments which encode mRNA expressing a biologically active novel vertebrate nuclear receptor which belongs to the nuclear receptor superfamily. A preferred embodiment relates to isolated nucleic acid fragments of SEQ ID NOs:1, 18, 19 and 20 which encode mRNA expressing a biologically functional derivative of human or mouse nNR5. Any such nucleic acid fragment will encode either a protein or protein fragment comprising at least an intracellular DNA-binding domain and/or ligand binding domain, domains conserved throughout the human nuclear receptor family domain which exist in nNR5 (SEQ ID NO:2, 21). Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists of nNR5.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant hosts, both prokaryotic and eukaryotic, which contain the substantially purified nucleic acid molecules disclosed throughout this specification.

A preferred embodiment of the present invention is an isolated cDNA molecule which encodes a human nuclear receptor protein, wherein said protein is substantially expressed in eye, especially the retina. The isolated cDNA molecules and expressed and isolated nuclear receptor proteins of the present invention are involved in the regulation of gene expression. Due to its high expression in retinal tissue, nNR5 should play an important role in eye function. Therapeutic compounds may be selected which interact with and regulate nNR5 activity in retina tissue which may be involved with diseases of the eye, including but not limited to cataracts and glaucoma, as well as retina-specific diseases such as diabetes mellitus, retinitis pigmentosa, macular degeneration, retinal detachment and retinoblastoma.

An especially preferred embodiment of the present invention is disclosed in FIGS. 1A-B and SEQ ID NO: 1, an isolated human cDNA encoding a novel nuclear trans-acting receptor protein, nNR5.

Another preferred aspect of the present invention relates to a substantially purified form of the novel nuclear trans-acting receptor protein, nNR5, which is disclosed in FIGS. 2A-B and FIG. 3 and as set forth in SEQ ID NO:2.

Another embodiment of the present invention relates to an isolated cDNA molecule encoding nNR5 which also contains a single intron from nucleotide #971 to nucleotide #1847 of SEQ ID NO: 18.

Another embodiment of the present invention relates to an isolated cDNA which contains the additional 70 nucleotides as disclosed in SEQ ID NO:18 in conjunction with the intron-less DNA sequence disclosed for SEQ ID NO:1, resulting in a DNA molecule disclosed herein as SEQ ID NO: 19.

The present invention also relates to biologically functional derivatives of nNR5 as set forth as SEQ ID NO:2, including but not limited to nNR5 mutants and biologically active fragments such as amino acid substitutions, deletions, additions, amino terminal truncations and carboxy-terminal truncations, such that these fragments provide for proteins or protein fragments of diagnostic, therapeutic or prophylactic use and would be useful for screening for agonists and/or antagonists of nNR5 function.

Another especially preferred embodiment of the present invention is disclosed in FIGS. 4A-B and SEQ ID NO:20, an isolated mouse cDNA encoding a novel nuclear trans-acting receptor protein, mouse nNR5.

Another preferred aspect of the present invention relates to a substantially purified form of the novel mouse nuclear trans-acting receptor protein, mouse nNR5, which is disclosed in FIGS. 5A-C and FIG. 6 and as set forth in SEQ ID NO:21.

The present invention also relates to a non-human transgenic animal which is heterozygous for a functional nNR5 gene native to that animal. In another embodiment, the animal of this aspect is used in a method to prepare an animal which expresses a non-native nNR5 gene in the absence of the expression of a native nNR5 gene. In particular embodiments the non-human animal is a mouse. In further embodiments the non-native nNR5 is a wild-type human nNR5 which is disclosed herein, or any other biologically equivalent form of human nNR5 gene as also disclosed herein.

Another aspect of the invention is a non-human animal embryo deficient for native nNR5 expression.

The present invention also relates to polyclonal and monoclonal antibodies raised in response to either the human or mouse form of nNR5 disclosed herein, or a biologically functional derivative thereof. It will be especially preferable to raise antibodies against epitopes within the NH₂-terminal domain of nNR5, which show the least homology to other known proteins belonging to the human nuclear receptor superfamily. To this end, the DNA molecules, RNA molecules, recombinant protein and antibodies of the present invention may be used to screen and measure levels of human nNR5. The recombinant proteins, DNA molecules, RNA molecules and antibodies lend themselves to the formulation of kits suitable for the detection and typing of human nNR5.

The present invention also relates to isolated nucleic acid molecules which are fusion constructions expressing fusion proteins useful in assays to identify compounds which modulate wild-type human and/or mouse nNR5 activity. A preferred aspect of this portion of the invention includes, but is not limited to, glutathione S-transferase GST-nNR5 fusion constructs. These fusion constructs include, but are not limited to, all or a portion of the ligand-binding domain of nNR5, respectively, as an in-frame fusion at the carboxy terminus of the GST gene. The disclosure of SEQ ID NOS:1, 2 and 18-21 allow the artisan of ordinary skill to construct any such nucleic acid molecule encoding a GST-nuclear receptor fusion protein. Soluble recombinant GST-nuclear receptor fusion proteins may be expressed in various expression systems, including Spodoptera frugiperda (Sf21) insect cells (Invitrogen) using a baculovirus expression vector (e.g., Bac-N-Blue DNA from Invitrogen or pAcG2T from Pharmingen). Additional preferred fusion constructs are detailed in Example 3.

It is an object of the present invention to provide an isolated nucleic acid molecule which encodes a novel form of a nuclear receptor protein such as human nNR5, human nuclear receptor protein fragments of full length proteins such as nNR5, and mutants which are derivatives of SEQ ID NO:2. Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR5 function.

It is also an object of the present invention to provide an isolated nucleic acid molecule which encodes a novel form of a nuclear receptor protein from mouse nNR5, mouse nuclear receptor protein fragments of full length proteins such as nNR5, and mutants which are derivatives of SEQ ID NO:21. Again, any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR5 function.

Another object of this invention is tissue typing using probes or antibodies of this invention. In a particular embodiment, polynucleotide probes are used to identify tissues expressing nNR5 mRNA. In another embodiment, probes or antibodies can be used to identify a type of tissue based on nNR5 expression or display of nNR5 receptors.

It is a further object of the present invention to provide the human and/or mouse nuclear receptor proteins or protein fragments encoded by the nucleic acid molecules referred to in the preceding paragraphs.

It is a further object of the present invention to provide recombinant vectors and recombinant host cells which comprise a nucleic acid sequence encoding human and/or mouse nNR5 or a biological equivalent thereof.

It is an object of the present invention to provide a substantially purified form of nNR5, as set forth in SEQ ID NO:2.

It is also an object of the present invention to provide a substantially purified form of nNR5, as set forth in SEQ ID NO:21.

It is an object of the present invention to provide for biologically functional derivatives of nNR5, including but not necessarily limited to amino acid substitutions, deletions, additions, amino terminal truncations and carboxy-terminal truncations such that these fragment and/or mutants provide for proteins or protein fragments of diagnostic, therapeutic or prophylactic use.

It is also an object of the present invention to provide for nNR5-based in-frame fusion constructions, methods of expressing these fusion constructions and biological equivalents disclosed herein, related assays, recombinant cells expressing these constructs and agonistic and/or antagonistic compounds identified through the use of DNA molecules encoding human and/or mouse nuclear receptor proteins such as nNR5.

As used herein, “DBD” refers to—DNA binding domain—

As used herein, “LBD” refers to—ligand binding domain—

As used herein, “KO” refers to—knockout mouse—

As used herein, the term “mammalian host” refers to any mammal, including a human being.

### BRIEF DESCRIPTION OF THE DRAWINGS

FIGS. 1A-B shows the nucleotide sequence (SEQ ID NO: 1) which comprises the open reading frame encoding the human nuclear receptor protein, nNR5.

FIGS. 2A-B shows the coding strand of the isolated cDNA molecule (SEQ ID NO: 1) which encodes human nNR5, and the amino acid sequence (SEQ ID NO: 2) of human nNR5. The region in bold is the DNA binding domain.

FIG. 3 shows the amino acid sequence (SEQ ID NO: 2) of human nNR5. The region in bold is the DNA binding domain.

FIGS. 4A-B shows the nucleotide sequence (SEQ ID NO:20) which comprises the open reading frame encoding the mouse nuclear receptor protein, nNR5.

FIGS. 5A-C shows the coding (SEQ ID NO:20) and anticoding (SEQ ID NO:22) strands which comprises the open reading frame for the mouse nuclear receptor (SEQ ID NO:21).

FIG. 6 shows the amino acid sequence (SEQ ID NO: 21) of mouse nNR5. The region in bold is the DNA binding domain, from Cys-39 to Met-106.

FIG. 7 shows the polypeptide alignment between mouse (comprised within SEQ ID NO:21) and human (SEQ ID NO:2) nuclear receptor 5 (nNR5). The overlapping region has 90% amino acid sequence identity. The DBD (DNA binding domains) are in bold, from Cys-39 to Met-106 of SEQ ID NO:21, and from Cys-47 to Met-113 of SEQ ID NO:2.

FIG. 8 shows Northern analysis on human brain, retina and human RPE (retina pigment epithelium) cells using a DNA probe corresponding to the whole LBD of human nNR5. The result shows that there are three mRNA bands detected only in the human retina sample. Each band corresponds to different splice variant. The ˜2.0 Kb band is the major transcript which encodes the 410 amino acid protein. The ˜7.0 Kb band encodes the 367 amino acid peptide disclosed as SEQ ID NO:2. β-actin detection showed that all the lanes had equal amount of mRNA.

FIG. 9, Panels a-h show in situ hybridization on both mouse and rhesus monkey retina. Mouse probes in the LBD region and 3′-UTR provide the same results. Panel a is the whole mouse eye section. The blue staining (arrows in black and white drawing) indicates Muller Glial cells (inner layer, arrows in black and white drawing) and RPE cells (the thin outer layer, arrows in black and white drawing). Panel b, c and d are immunohistochemistry staining using Muller Glial cell specific markers—S100 and GFAP. The signal in red (light shade in black and white drawing) indicates Muller cells and its processes. Panel e is an amplification section of mouse retina. The black stain (bracketed regions in black and white drawing) show expression of mouse nNR5 in Muller layer (top) and RPE layer (bottom). The dotted signals were nNR5 expression in Muller cell processes. Panel f is a mouse retina section hybridized with sense probe which is a negative control. No signal was detected in negative control (as indicated by a lack of bracketing in Panel f). Panel h and i are rhesus monkey retina sections. The same result is presented in two different image systems. In panel h, the blue fluorescent (light shaded portion of bracketed region in black and white drawing) shows the expression of nNR5, while in panel i, the expression level is in pseudo color. Yellow to green color indicated higher level of mRNA expression. The bracketed region of Panel h in the black and white drawing shows expression levels corresponding to red and yellow regions. The human result confirmed the mouse data showing nNR5 expression in Muller Glial and RPE cells.

FIGS. 10A-C show results from a receptor/reporter co-transfection assay. A Ga14-nNR5-LBD construct and SEAP reporter with Ga14 binding sites were cotransfected in either CV1 or RPE cells. FIG. 10A shows that increasing amount of Ga14-nNR5-LBD reduces SEAP expression level. This reduction is contributed through inhibiting promoter activity and squelching of transcription factors because partial inhibition is also observed on SEAP reporter without Ga14 binding sites (SEAP-GS). A CMV vector containing Ga14 domain did not contribute to the inhibition. Same results were obtained on RPE-J cells (FIG. 10B). FIG. 10C demonstrates that the inhibition function of nNR5-LBD is not observed on other nuclear receptors, such as, Erb-LBD or hPXR-LBD.

### DETAILED DESCRIPTION OF THE INVENTION

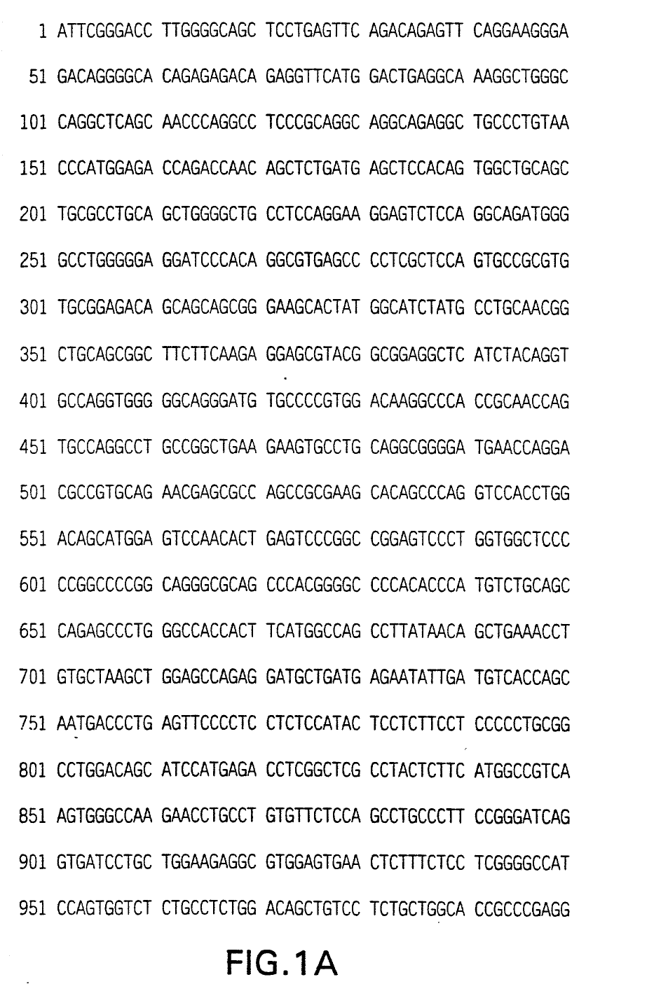

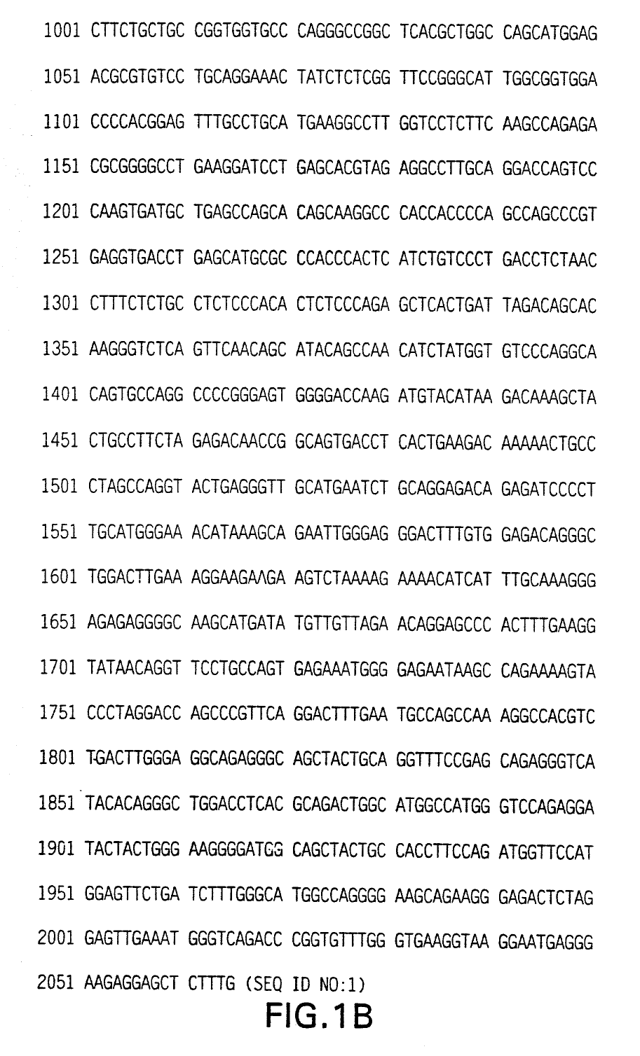

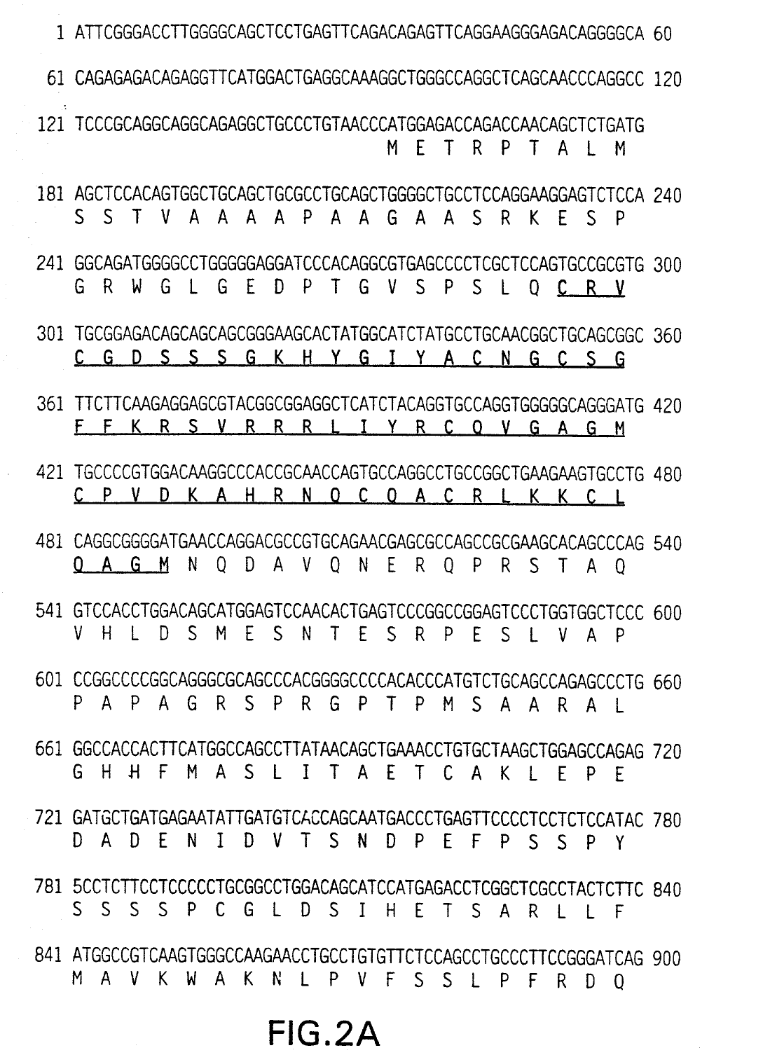

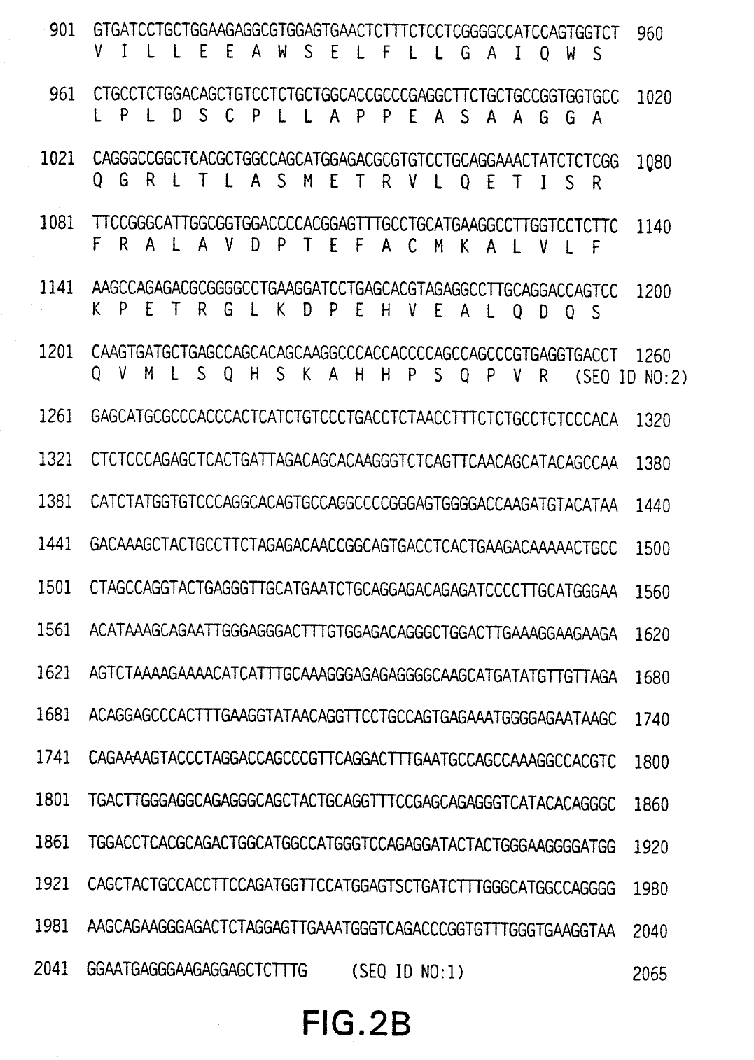

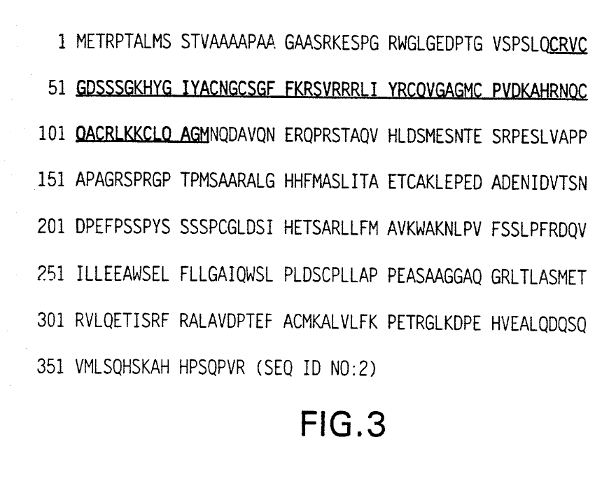

The present invention relates to isolated nucleic acid and protein forms which represent nuclear receptors, preferably but not necessarily limited to human receptors or mouse receptors. These expressed proteins are novel nuclear receptors and which are useful in the identification of downstream target genes and ligands regulating their activity. The nuclear receptor proteins encoded by the isolated polynucleotides of the present invention are involved in the regulation of in vivo cell proliferation and/or cell development. The nuclear receptor superfamily is composed of a group of structurally related receptors which are regulated by chemically distinct ligands. The common structure for a nuclear receptor is a highly conserved DNA binding domain (DBD) located in the center of the peptide and the ligand-binding domain (LBD) at the COOH-terminus. Eight out of the nine non-variant cysteines form two type II zinc fingers which distinguish nuclear receptors from other DNA-binding proteins. The DBDs share at least 50% to 60% amino acid sequence identity even among the most distant members in vertebrates. The superfamily has been expanded within the past decade to contain approximately 25 subfamilies. An EST database search using whole peptide sequences of several representative subfamily members, were utilized to identify a human EST (GenBank Acc. No. W27871; dbEST Id #534939; search available through National Center for Biotechnology Information—http://www.ncbi.nlm.nih.gov/dbEST/index.html) which encodes a portion of a novel member of the nuclear receptor superfamily. In addition, the exemplified cDNA encoding nNR5 was isolated using DNA fragments encoding DBD regions of androgen receptor (AR), estrogen receptor b (ERb), glucocorticoid receptor (GR) and vitamin D receptor (VDR) as probes to screen a human retina cDNA library and a library made from mRNA derived from 20 major human tissues commercially available from Clontech (Palo Alto, Calif.) at low stringency. Twenty positive clones were obtained by screening 250,000 primary clones from a human retina cDNA library constructed in the lab. Sequence information was obtained by directly sequencing one of the purified clones (FIGS. 1A-B; SEQ ID NO: 1). A peptide of 367 amino acids encoded by the cDNA has the authentic domain structures of the nuclear receptor (FIGS. 2A-B, FIG. 3; SEQ ID NO: 2). A data base search revealed that two other ESTs from a retina library matching this clone in non-conserved region in addition to EST W27871, which are Gen Bank Acc. No. W21793 (dbEST Id†534939; http://www.ncbi.nln.nih.gov/dbEST/index.html) and Gen Bank Acc. No. W21801 (dbEST Id#534939; http://www.ncbi.nlm.nih.gov/dbEST/index.html). A known gene which is most related to nNR5 at peptide sequence level is chicken ovalbumin upstream promoter transcription factor (COUP-TF). The protein nNR5 is 43% homologous in overlapping regions to COUP-TF. The gene encoding human nNR5 is located on chromosome 15. Expression of human nNR5 was not detected in the majority of the tissues examined via RT-PCR, but it is very abundant in retina based on screening results and northern analysis. Therefore, nNR5 represents a new subfamily of the nuclear receptor superfamily because its low homology to other members in the superfamily. As noted above, due to its high expression in retinal tissue, nNR5 should play an important role in eye function. Therapeutic compounds may be selected which interact with and regulate nNR5 activity in retina tissue which may be involved with diseases of the eye, including but not limited to cataracts and glaucoma, as well as retina-specific diseases such as diabetes mellitus, retinitis pigmentosa, macular degeneration, retinal detachment and retinablastoma.

The present invention also relates to isolated nucleic acid fragments of nNR5 (SEQ ID NO: 1) which encode mRNA expressing a biologically active novel human nuclear receptor. Any such nucleic acid fragment will encode either a protein or protein fragment comprising at least an intracellular DNA-binding domain and/or ligand binding domain, domains conserved throughout the human nuclear receptor family domain which exist in nNR5 (SEQ ID NO:2). Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR5 function.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant hosts, both prokaryotic and eukaryotic, which contain the substantially purified nucleic acid molecules disclosed throughout this specification.

A preferred aspect of the present invention is disclosed in FIGS. 1A-B and SEQ ID NO: 1, a human cDNA encoding a novel nuclear trans-acting receptor protein, nNR5, disclosed as follows:

(SEQ ID NO:1)
ATTCGGGACC TTGGGGCAGC TCCTGAGTTC AGACAGAGTT
CAGGAAGGGA
GACAGGGGCA CAGAGAGACA GAGGTTCATG GACTGAGGCA
AAGGCTGGGC
CAGGCTCAGC AACCCAGGCC TCCCGCAGGC AGGCAGAGGC
TGCCCTGTAA
CCCATGGAGA CCAGACCAAC AGCTCTGATG AGCTCCACAG
TGGCTGCAGC
TGCGCCTGCA GCTGGGGCTG CCTCCAGGAA GGAGTCTCCA
GGCAGATGGG
GCCTGGGGGA GGATCCCACA GGCGTGAGCC CCTCGCTCCA
GTGCCGCGTG
TGCGGAGACA GCAGCAGCGG GAAGCACTAT GGCATCTATG
CCTGCAACGG
CTGCAGCGGC TTCTTCAAGA GGAGCGTACG GCGGAGGCTC
ATCTACAGGT
GCCAGGTGGG GGCAGGGATG TGCCCCGTGG ACAAGGCCCA
CCGCAACCAG
TGCCAGGCCT GCCGGCTGAA GAAGTGCCTG CAGGCGGGGA
TGAACCAGGA
CGCCGTGCAG AACGAGCGCC AGCCGCGAAG CACAGCCCAG
GTCCACCTGG
ACAGCATGGA GTCCAACACT GAGTCCCGGC CGGAGTCCCT
GGTGGCTCCC
CCGGCCCCGG CAGGGCGCAG CCCACGGGGC CCCACACCCA
TGTCTGCAGC
CAGAGCCCTG GGCCACCACT TCATGGCCAG CCTTATAACA
GCTGAAACCT
GTGCTAAGCT GGAGCCAGAG GATGCTGATG AGAATATTGA
TGTCACCAGC
AATGACCCTG AGTTCCCCTC CTCTCCATAC TCCTCTTCCT
CCCCCTGCGG
CCTGGACAGC ATCCATGAGA CCTCGGCTCG CCTACTCTTC
ATGGCCGTCA
AGTGGGCCAA GAACCTGCCT GTGTTCTCCA GCCTGCCCTT
CCGGGATCAG
GTGATCCTGC TGGAAGAGGC GTGGAGTGAA CTCTTTCTCC
TCGGGGCCAT
CCAGTGGTCT CTGCCTCTGG ACAGCTGTCC TCTGCTGGCA
CCGCCCGAGG
CTTCTGCTGC CGGTGGTGCC CAGGGCCGGC TCACGCTGGC
CAGCATGGAG
ACGCGTGTCC TGCAGGAAAC TATCTCTCGG TTCCGGGCAT
TGGCGGTGGA
CCCCACGGAG TTTGCCTGCA TGAAGGCCTT GGTCCTCTTC
AAGCCAGAGA
CGCGGGGCCT GAAGGATCCT GAGCACGTAG AGGCCTTGCA
GGACCAGTCC
CAAGTGATGC TGAGCCAGCA CAGCAAGGCC CACCACCCCA
GCCAGCCCGT
GAGGTGACCT GAGCATGCGC CCACCCACTC ATCTGTCCCT
GACCTCTAAC
CTTTCTCTGC CTCTCCCACA CTCTCCCAGA GCTCACTGAT
TAGACAGCAC
AAGGGTCTCA GTTCAACAGC ATACAGCCAA CATCTATGGT
GTCCCAGGCA
CAGTGCCAGG CCCCGGGAGT GGGGACCAAG ATGTACATAA
GACAAAGCTA
CTGCCTTCTA GAGACAACCG GCAGTGACCT CACTGAAGAC
AAAAACTGCC
CTAGCCAGGT ACTGAGGGTT GCATGAATCT GCAGGAGACA
GAGATCCCCT
TGCATGGGAA ACATAAAGCA GAATTGGGAG GGACTTTGTG
GAGACAGGGC
TGGACTTGAA AGGAAGAAGA AGTCTAAAAG AAAACATCAT
TTGCAAAGGG
AGAGAGGGGC AAGCATGATA TGTTGTTAGA ACAGGAGCCC
ACTTTGAAGG
TATAACAGGT TCCTGCCAGT GAGAAATGGG GAGAATAAGC
CAGAAAAGTA
CCCTAGGACC AGCCCGTTCA GGACTTTGAA TGCCAGCCAA
AGGCCACGTC
TGACTTGGGA GGCAGAGGGC AGCTACTGCA GGTTTCCGAG
CAGAGGGTCA
TACACAGGGC TGGACCTCAC GCAGACTGGC ATGGCCATGG
GTCCAGAGGA
TACTACTGGG AAGGGGATGG CAGCTACTGC CACCTTCCAG
ATGGTTCCAT
GGAGTTCTGA TCTTTGGGCA TGGCCAGGGG AAGCAGAAGG
GAGACTCTAG
GAGTTGAAAT GGGTCAGACC CGGTGTTTGG GTGAAGGTAA
GGAATGAGGG

AAGAGGAGCT CTTTG.

The present invention also relates to a substantially purified form of the novel nuclear trans-acting receptor protein, nNR5, which is shown in FIGS. 2A-B and FIG. 3 and as set forth in SEQ ID NO:2, disclosed as follows:

(SEQ ID NO:2)
METRPTALMS STVAAAAPAA GAASRKESPG RWGLGEDPTG
VSPSLQCRVC
GDSSSGKHYG IYACNGCSGF FKRSVRRRLI YRCQVGAGMC
PVDKAHRNQC
QACRLKKCLQ AGMNQDAVQN ERQPRSTAQV HLDSMESNTE
SRPESLVAPP
APAGRSPRGP TPMSAARALG HHFMASLITA ETCAKLEPED
ADENIDVTSN
DPEFPSSPYS SSSPCGLDSI HETSARLLFM AVKWAKNLPV
FSSLPFRDQV
ILLEEAWSEL FLLGAIQWSL PLDSCPLLAP PEASAAGGAQ
GRLTLASMET
RVLQETISRF RALAVDPTEF ACMKALVLFK PETRGLKDPE
HVEALQDQSQ

VMLSQHSKAH HPSQPVR.

SEQ ID NO:1 contains 2056 nucleotides. This DNA molecule contains an open reading frame from nucleotide 154 (initiating Met from nt 154-156) to nucleotide 1254, with a “TGA” termination codon from nucleotides 1255-1257. This open reading frame encodes the 367 amino acid human nNR5 protein as set forth in SEQ ID NO:2.

The present invention also relates to biologically functional derivatives and/or mutants of nNR5 as set forth as SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR5 function.

The present invention also relates to an isolated cDNA molecule which comprises the nucleotide sequence which encodes the entire reading frame of human NR5, as well as containing an intron, from nucleotide 971 to nucleotide 1847, as underlined below and as set forth as SEQ ID NO: 18:

| | |
|---|---|
| TATAGGGCGA ATTGGGTACC GGGCCCCCCC TCGAGGTCGA CGGTATCGAT | (SEQ ID NO:18) |
| AAGCTTGATA TCGAATTCGA ATTCGGGACC TTGGGGCAGC TCCTGAGTTC | |
| AGACAGAGTT CAGGAAGGGA GACAGGGGCA CAGAGAGACA GAGGTTCATG | |
| GACTGAGGCA AAGGCTGGGC CAGGCTCAGC AACCCAGGCC TCCCGCAGGC | |
| AGGCAGAGGC TGCCCTGTAA CCCATGGAGA CCAGACCAAC AGCTCTGATG | |
| AGCTCCACAG TGGCTGCAGC TGCGCCTGCA GCTGGGGCTG CCTCCAGGAA | |
| GGAGTCTCCA GGCAGATGGG GCCTGGGGGA GGATCCCACA GGCGTGAGCC | |
| CCTCGCTCCA GTGCCGCGTG TGCGGAGACA GCAGCAGCGG GAAGCACTAT | |
| GGCATCTATG CCTGCAACGG CTGCAGCGGC TTCTTCAAGA GGAGCGTACG | |
| GCGGAGGCTC ATCTACAGGT GCCAGGTGGG GGCAGGGATG TGCCCCGTGG | |
| ACAAGGCCCA CCGCAACCAG TGCCAGGCCT GCCGGCTGAA GAAGTGCCTG | |
| CAGGCGGGGA TGAACCAGGA CGCCGTGCAG AACGAGCGCC AGCCGCGAAG | |
| CACAGCCCAG GTCCACCTGG ACAGCATGGA GTCCAACACT GAGTCCCGGC | |
| CGGAGTCCCT GGTGGCTCCC CCGGCCCCGG CAGGGCGCAG CCCACGGGGC | |
| CCCACACCCA TGTCTGCAGC CAGAGCCCTG GGCCACCACT TCATGGCCAG | |
| CCTTATAACA GCTGAAACCT GTGCTAAGCT GGAGCCAGAG GATGCTGATG | |
| AGAATATTGA TGTCACCAGC AATGACCCTG AGTTCCCCTC CTCTCCATAC | |
| TCCTCTTCCT CCCCCTGCGG CCTGGACAGC ATCCATGAGA CCTCGGCTCG | |
| CCTACTCTTC ATGGCCGTCA AGTGGGCCAA GAACCTGCCT GTGTTCTCCA | |
| GCCTGCCCTT CCGGGATCAG GTACCTACCG GCCTGCCTGC TGGGGAGCTA | |
| GGCTGGGCTG GGGTCAGGCG GCCCACTCGA GTCAACCAGA CAGGGCACAC | |
| ACATCCCCAC GCCAGTATGA ATGCACACAG CTTGGATGGT GATGGCTGGG | |
| GACACACATA CCTCTGATTC AGCGATGGCT GGGGTGCATC TCAGGGATGG | |
| TGACGGTGGG GGTGCATGCA TCTCTGGCAC AGGGATGATG GTCGGGGTGC | |
| ACACCTAGGA GATGATGATG GCTAGGGACC TACAGGGCCC AGGGTCTTCT | |
| TAAGTTCTGG AAGACCCTCA GGCCCTGCAG ACATTCTGTG GGTAACAAGT | |
| GACCTGCACA CCCTGAACAG GCTGAGTGGC TGACTCTAGG CCCCCTTGGA | |
| GCACAAGTGC CTACGACTTC AGGGCTTGCA TTTTAGTTCA ATCTCTCCAG | |
| CTCTGGGCCA TCCCTCTCGG CTTCTAATGG GCAAGCAGAT CTTTCAGGAA | |
| AACCAGGAGG AGAGGCATGA GGAAGGTTTG AGGCCCTCAG CCAGTCTGTG | |
| TGCTGGGGTG GAGCAACTCA GAAGAGTCAG GCCACACCAC TTGAATACAC | |
| TCAACTTAGG ACACTCATGA GGCATGTCTC TGAGGCTGCC CAACTTCCAA | |
| TGGCTCTGGG CGTTCCTAAA TGTCCCAGCT GCAGCTCTGG ATGGAACCCA | |
| GTGTCTCAGA TGATAGGCAG CTGAGCCGGA TGGTGCCAAA TCCCAGAGCT | |
| CTGAGCCTCT GGCTGATGTC AGGAGAGCAT TCTCGGGTCC CAGGACAGCA | |
| CTTCCATTCC TTGGGTGCCT GAGATGGTGG CAGAGGCTCC AGACTGAGCC | |
| AGAGAAGCTG TGTGTCTGCC ATAACAGGCA CCCCTGTCTG AGCACAGGTG | |
| ATCCTGCTGG AAGAGGCGTG GAGTGAACTC TTTCTCCTCG GGGCCATCCA | |
| GTGGTCTCTG CCTCTGGACA GCTGTCCTCT GCTGGCACCG CCCGAGGCCT | |
| CTGCTGCCGG TGGTGCCCAG GGCCGGCTCA CGCTGGCCAG CATGGAGACG | |
| CGTGTCCTGC AGGAAACTAT CTCTCGGTTC CGGGCATTGG CGGTGGACCC | |
| CACGGAGTTT GCCTGCATGA AGGCCTTGGT CCTCTTCAAG CCAGAGACGC | |
| GGGGCCTGAA GGATCCTGAG CACGTAGAGG CCTTGCAGGA CCAGTCCCAA | |
| GTGATGCTGA GCCAGCACAG CAAGGCCCAC CACCCCAGCC AGCCCGTGAG | |
| GTGACCTGAG CATGCGCCCA CCCACTCATC TGTCCCTGAC CTCTAACCTT | |
| TCTCTGCCTC TCCCACACTC TCCCAGAGCT CACTGATTAG ACAGCACAAG | |
| GGTCTCAGTT CAACAGCATA CAGCCAACAT CTATGGTGTC CCAGGCACAG | |
| TGCCAGGCCC CGGGAGTGGG GACCAAGATG TACATAAGAC AAAGCTACTG | |
| CCTTCTAGAG ACAACCGGCA GTGACCTCAC TGAAGACAAA AACTGCCCTA | |
| GCCAGGTACT GAGGGTTGCA TGAATCTGCA GGAGACAGAG ATCCCCTTGC | |
| ATGGGAAACA TAAAGCAGAA TTGGGAGGGA CTTTGTGGAG ACAGGGCTGG | |
| ACTTGAAAGG AAGAAGAAGT CTAAAAGAAA ACATCATTTG CAAAGGGAGA | |
| GAGGGGCAAG CATGATATGT TGTTAGAACA GGAGCCCACT TTGAAGGTAT | |
| AACAGGTTCC TGCCAGTGAG AAATGGGGAG AATAAGCCAG AAAAGTACCC | |
| TAGGACCAGC CCGTTCAGGA CTTTGAATGC CAGCCAAAGG CCACGTCTGA | |
| CTTGGGAGGC AGAGGGCAGC TACTGCAGGT TTCCGAGCAG AGGGTCATAC | |
| ACAGGGCTGG ACCTCACGCA GACTGGCATG GCCATGGGTC CAGAGGATAC | |
| TACTGGGAAG GGGATGGCAG CTACTGCCAC CTTCCAGATG GTTCCATGGA | |
| GTTCTGATCT TTGGGCATGG CCAGGGGAAG CAGAAGGGAG ACTCTAGGAG | |
| TTGAAATGGG TCAGACCCGG TGTTTGGGTG AAGGTAAGGA ATGAGGGAAG | |
| AGGAGCTCTT TG. | |

The intron-containing nNR5 cDNA as set forth in SEQ ID NO: 18 contains an additional 70 nucleotides at the 5′ end of the clone. Therefore, the present invention also relates to an isolated cDNA which comprises the open reading frame of SEQ ID NO:1, in addition to the additional 70 nucleotides at the 5′ end of an isolated polynucleotide encoding nNR5. This nucleotide sequence is shown below and is as set forth in SEQ ID NO:19:

(SEQ ID NO:19)
TATAGGGCGA ATTGGGTACC GGGCCCCCCC TCGAGGTCGA
CGGTATCGAT
AAGCTTGATA TCGAATTCGA ATTCGGGACC TTGGGGCAGC
TCCTGAGTTC
AGACAGAGTT CAGGAAGGGA GACAGGGGCA CAGAGAGACA
GAGGTTCATG
GACTGAGGCA AAGGCTGGGC CAGGCTCAGC AACCCAGGCC
TCCCGCAGGC
AGGCAGAGGC TGCCCTGTAA CCCATGGAGA CCAGACCAAC
AGCTCTGATG
AGCTCCACAG TGGCTGCAGC TGCGCCTGCA GCTGGGGCTG
CCTCCAGGAA
GGAGTCTCCA GGCAGATGGG GCCTGGGGGA GGATCCCACA
GGCGTGAGCC
CCTCGCTCCA GTGCCGCGTG TGCGGAGACA GCAGCAGCGG
GAAGCACTAT
GGCATCTATG CCTGCAACGG CTGCAGCGGC TTCTTCAAGA
GGAGCGTACG
GCGGAGGCTC ATCTACAGGT GCCAGGTGGG GGCAGGGATG
TGCCCCGTGG
ACAAGGCCCA CCGCAACCAG TGCCAGGCCT GCCGGCTGAA
GAAGTGCCTG
CAGGCGGGGA TGAACCAGGA CGCCGTGCAG AACGAGCGCC
AGCCGCGAAG
CACAGCCCAG GTCCACCTGG ACAGCATGGA GTCCAACACT
GAGTCCCGGC
CGGAGTCCCT GGTGGCTCCC CCGGCCCCGG CAGGGCGCAG
CCCACGGGGC
CCCACACCCA TGTCTGCAGC CAGAGCCCTG GGCCACCACT
TCATGGCCAG
CCTTATAACA GCTGAAACCT GTGCTAAGCT GGAGCCAGAG
GATGCTGATG
AGAATATTGA TGTCACCAGC AATGACCCTG AGTTCCCCTC
CTCTCCATAC
TCCTCTTCCT CCCCCTGCGG CCTGGACAGC ATCCATGAGA
CCTCGGCTCG
CCTACTCTTC ATGGCCGTCA AGTGGGCCAA GAACCTGCCT
GTGTTCTCCA
GCCTGCCCTT CCGGGATCAG GTGATCCTGC TGGAAGAGGC
GTGGAGTGAA
CTCTTTCTCC TCGGGGCCAT CCAGTGGTCT CTGCCTCTGG
ACAGCTGTCC
TCTGCTGGCA CCGCCCGAGG CCTCTGCTGC CGGTGGTGCC
CAGGGCCGGC
TCACGCTGGC CAGCATGGAG ACGCGTGTCC TGCAGGAAAC
TATCTCTCGG
TTCCGGGCAT TGGCGGTGGA CCCCACGGAG TTTGCCTGCA
TGAAGGCCTT
GGTCCTCTTC AAGCCAGAGA CGCGGGGCCT GAAGGATCCT
GAGCACGTAG
AGGCCTTGCA GGACCAGTCC CAAGTGATGC TGAGCCAGCA
CAGCAAGGCC
CACCACCCCA GCCAGCCCGT GAGGTGACCT GAGCATGCGC
CCACCCACTC
ATCTGTCCCT GACCTCTAAC CTTTCTCTGC CTCTCCCACA
CTCTCCCAGA
GCTCACTGAT TAGACAGCAC AAGGGTCTCA GTTCAACAGC
ATACAGCCAA
CATCTATGGT GTCCCAGGCA CAGTGCCAGG CCCCGGGAGT
GGGGACCAAG
ATGTACATAA GACAAAGCTA CTGCCTTCTA GAGACAACCG
GCAGTGACCT
CACTGAAGAC AAAAACTGCC CTAGCCAGGT ACTGAGGGTT
GCATGAATCT
GCAGGAGACA GAGATCCCCT TGCATGGGAA ACATAAAGCA
GAATTGGGAG
GGACTTTGTG GAGACAGGGC TGGACTTGAA AGGAAGAAGA
AGTCTAAAAG
AAAACATCAT TTGCAAAGGG AGAGAGGGGC AAGCATGATA
TGTTGTTAGA
ACAGGAGCCC ACTTTGAAGG TATAACAGGT TCCTGCCAGT
GAGAAATGGG
GAGAATAAGC CAGAAAAGTA CCCTAGGACC AGCCCGTTCA
GGACTTTGAA
TGCCAGCCAA AGGCCACGTC TGACTTGGGA GGCAGAGGGC
AGCTACTGCA
GGTTTCCGAG CAGAGGGTCA TACACAGGGC TGGACCTCAC
GCAGACTGGC
ATGGCCATGG GTCCAGAGGA TACTACTGGG AAGGGGATGG
CAGCTACTGC
CACCTTCCAG ATGGTTCCAT GGAGTTCTGA TCTTTGGGCA
TGGCCAGGGG
AAGCAGAAGG GAGACTCTAG GAGTTGAAAT GGGTCAGACC
CGGTGTTTGG

GTGAAGGTAA GGAATGAGGG AAGAGGAGCT CTTTG.

The present invention also relates to an isolated DNA molecule which encodes a mouse nuclear receptor protein. Degenerate oligonucleotide primers were designed from the human cDNA clone and used to perform PCR on a mouse eye cDNA library. PCR products were sequenced and a second round of PCR using primers derived from sequence information obtained was performed to isolate a PCR product representing the entire open reading frame of a cDNA molecule encoding a mouse nNR5 protein.

The present invention also relates to recombinant vectors and recombinant hosts, both prokaryotic and eukaryotic, which contain the substantially purified nucleic acid molecules disclosed throughout this specification, including but not limited to an isolated DNA molecule which encodes a mouse nNR5 or mouse nNR5-like protein.

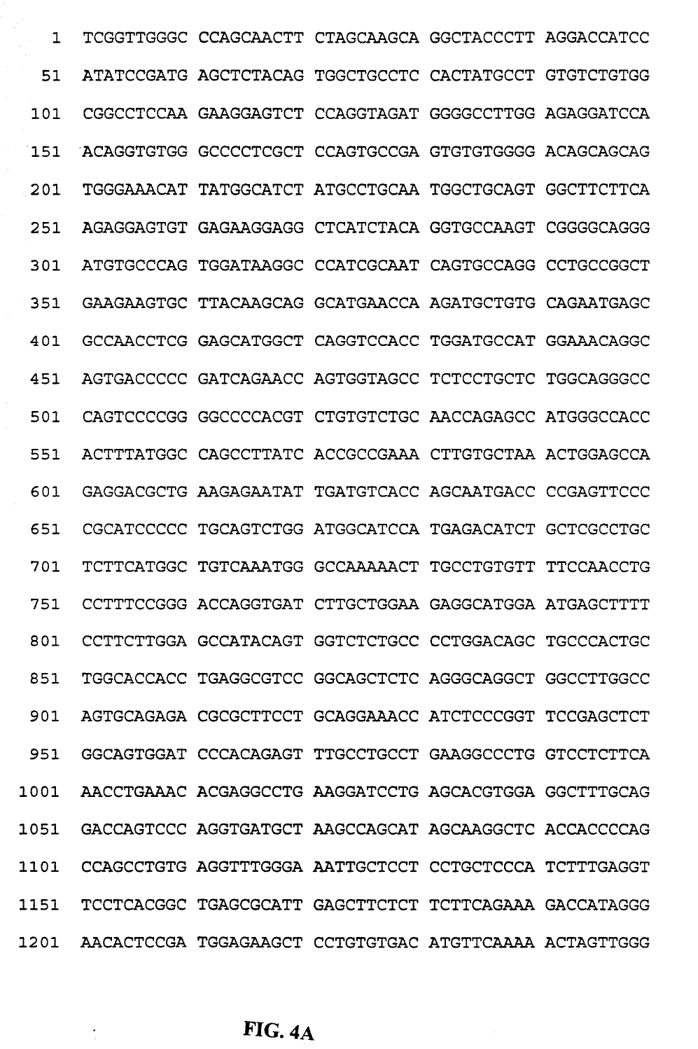

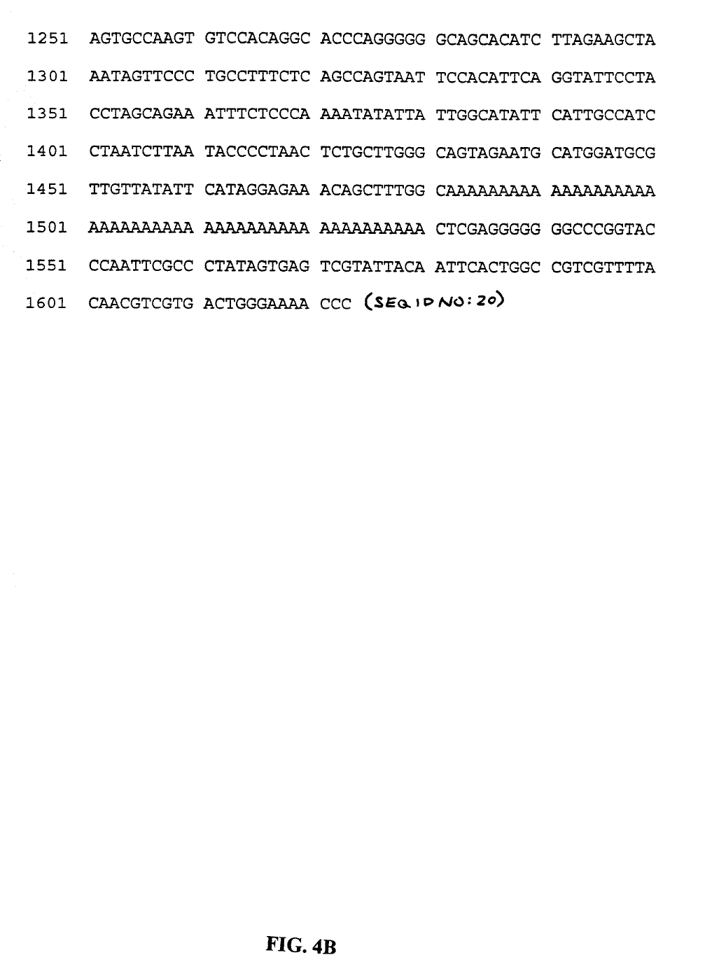

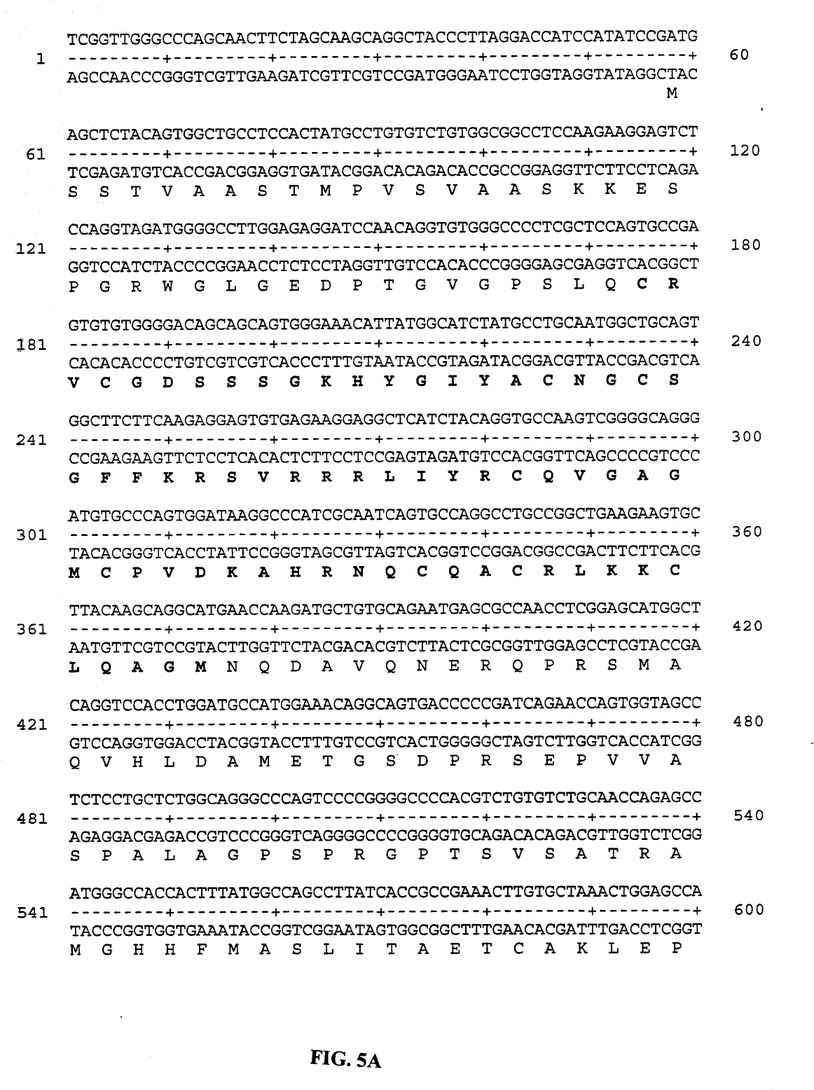

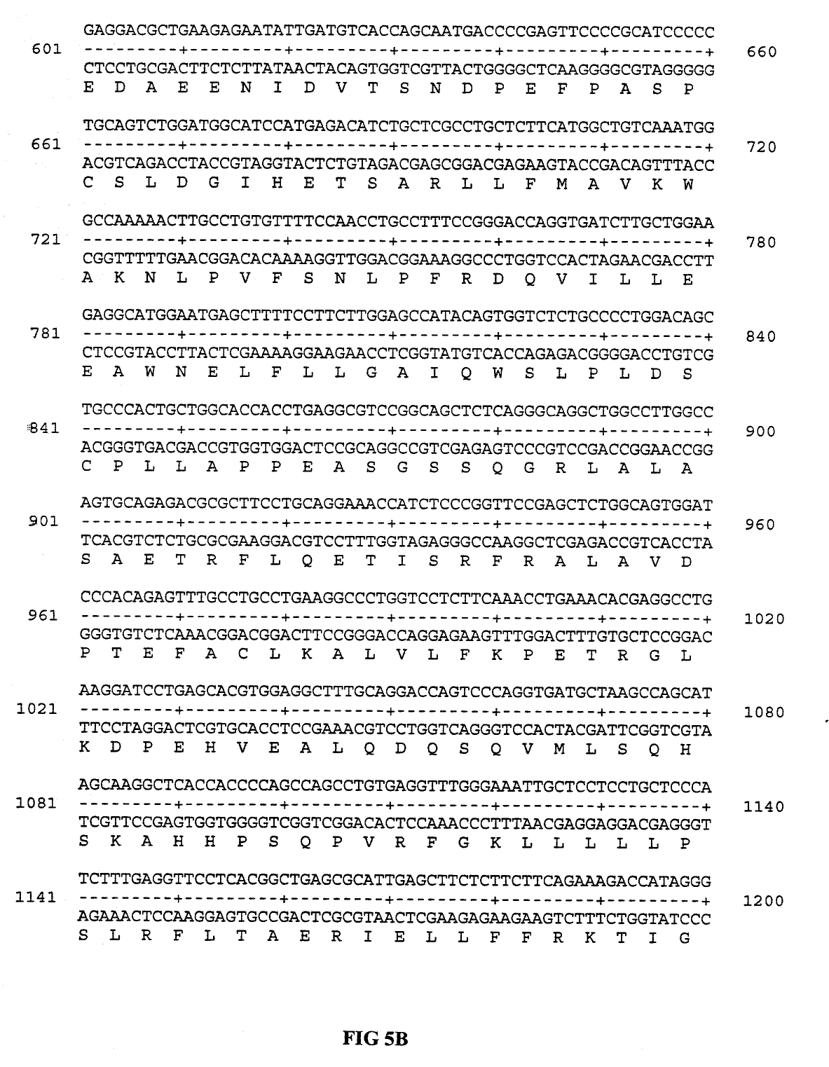

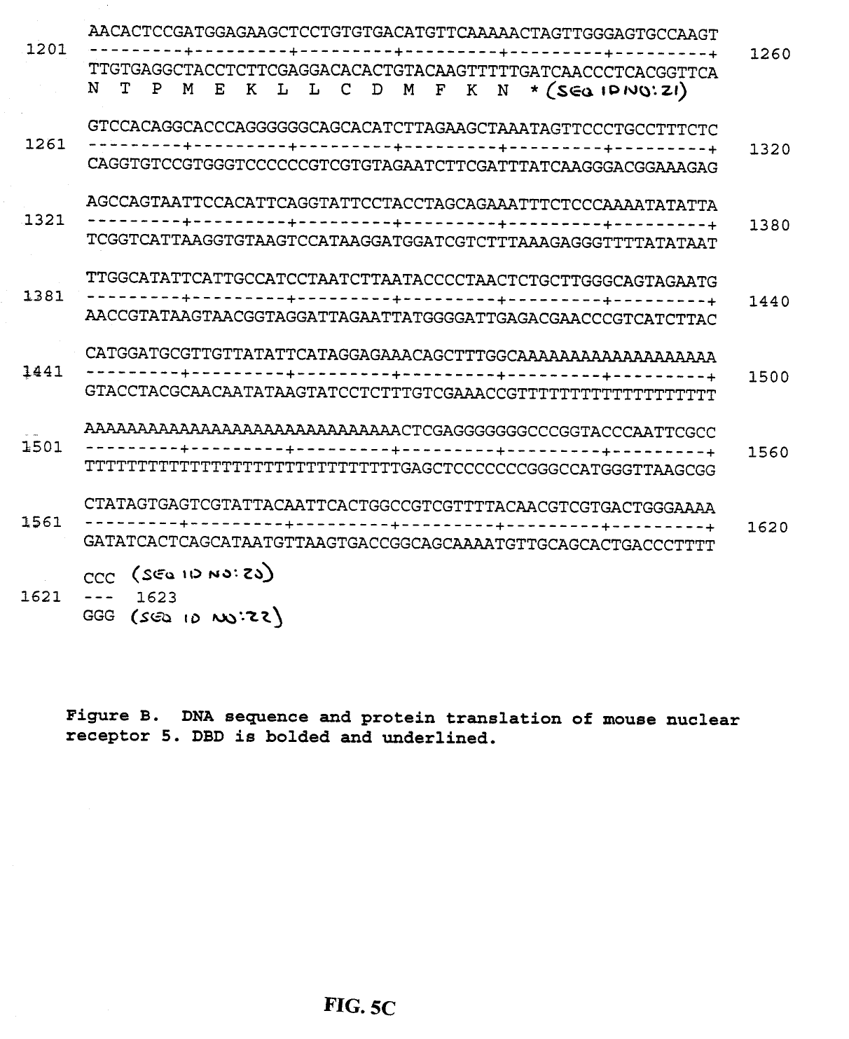

Another preferred aspect of the present invention is disclosed in FIGS. 4A-B, FIGS. 5A-C and SEQ ID NO:20, a cDNA encoding a novel mouse nuclear trans-acting receptor protein, nNR5, disclosed as follows:

(SEQ ID NO:20)
TCGGTTGGGC CCAGCAACTT CTAGCAAGCA GGCTACCCTT
AGGACCATCC
ATATCCGATG AGCTCTACAG TGGCTGCCTC CACTATGCCT
GTGTCTGTGG
CGGCCTCCAA GAAGGAGTCT CCAGGTAGAT GGGGCCTTGG
AGAGGATCCA
ACAGGTGTGG GCCCCTCGCT CCAGTGCCGA GTGTGTGGGG
ACAGCAGCAG
TGGGAAACAT TATGGCATCT ATGCCTGCAA TGGCTGCAGT
GGCTTCTTCA
AGAGGAGTGT GAGAAGGAGG CTCATCTACA GGTGCCAAGT
CGGGGCAGGG
ATGTGCCCAG TGGATAAGGC CCATCGCAAT CAGTGCCAGG
CCTGCCGGCT
GAAGAAGTGC TTACAAGCAG GCATGAACCA AGATGCTGTG
CAGAATGAGC
GCCAACCTCG GAGCATGGCT CAGGTCCACC TGGATGCCAT
GGAAACAGGC
AGTGACCCCC GATCAGAACC AGTGGTAGCC TCTCCTGCTC
TGGCAGGGCC
CAGTCCCCGG GGCCCCACGT CTGTGTCTGC AACCAGAGCC
ATGGGCCACC
ACTTTATGGC CAGCCTTATC ACCGCCGAAA CTTGTGCTAA
ACTGGAGCCA
GAGGACGCTG AAGAGAATAT TGATGTCACC AGCAATGACC
CCGAGTTCCC
CGCATCCCCC TGCAGTCTGG ATGGCATCCA TGAGACATCT
GCTCGCCTGC
TCTTCATGGC TGTCAAATGG GCCAAAAACT TGCCTGTGTT
TTCCAACCTG
CCTTTCCGGG ACCAGGTGAT CTTGCTGGAA GAGGCATGGA
ATGAGCTTTT
CCTTCTTGGA GCCATACAGT GGTCTCTGCC CCTGGACAGC
TGCCCACTGC
TGGCACCACC TGAGGCGTCC GGCAGCTCTC AGGGCAGGCT
GGCCTTGGCC
AGTGCAGAGA CGCGCTTCCT GCAGGAAACC ATCTCCCGGT
TCCGAGCTCT
GGCAGTGGAT CCCACAGAGT TTGCCTGCCT GAAGGCCCTG
GTCCTCTTCA
AACCTGAAAC ACGAGGCCTG AAGGATCCTG AGCACGTGGA
GGCTTTGCAG
GACCAGTCCC AGGTGATGCT AAGCCAGCAT AGCAAGGCTC
ACCACCCCAG
CCAGCCTGTG AGGTTTGGGA AATTGCTCCT CCTGCTCCCA
TCTTTGAGGT
TCCTCACGGC TGAGCGCATT GAGCTTCTCT TCTTCAGAAA
GACCATAGGG
AACACTCCGA TGGAGAAGCT CCTGTGTGAC ATGTTCAAAA
ACTAGTTGGG
AGTGCCAAGT GTCCACAGGC ACCCAGGGGG GCAGCACATC
TTAGAAGCTA
AATAGTTCCC TGCCTTTCTC AGCCAGTAAT TCCACATTCA
GGTATTCCTA
CCTAGCAGAA ATTTCTCCCA AAATATATTA TTGGCATATT
CATTGCCATC
CTAATCTTAA TACCCCTAAC TCTGCTTGGG CAGTAGAATG
CATGGATGCG
TTGTTATATT CATAGGAGAA ACAGCTTTGG CAAAAAAAAA
AAAAAAAAAA
AAAAAAAAAA AAAAAAAAAA AAAAAAAAAA CTCGAGGGGG
GGCCCGGTAC
CCAATTCGCC CTATAGTGAG TCGTATTACA ATTCACTGGC
CGTCGTTTTA

CAACGTCGTG ACTGGGAAAA CCC.

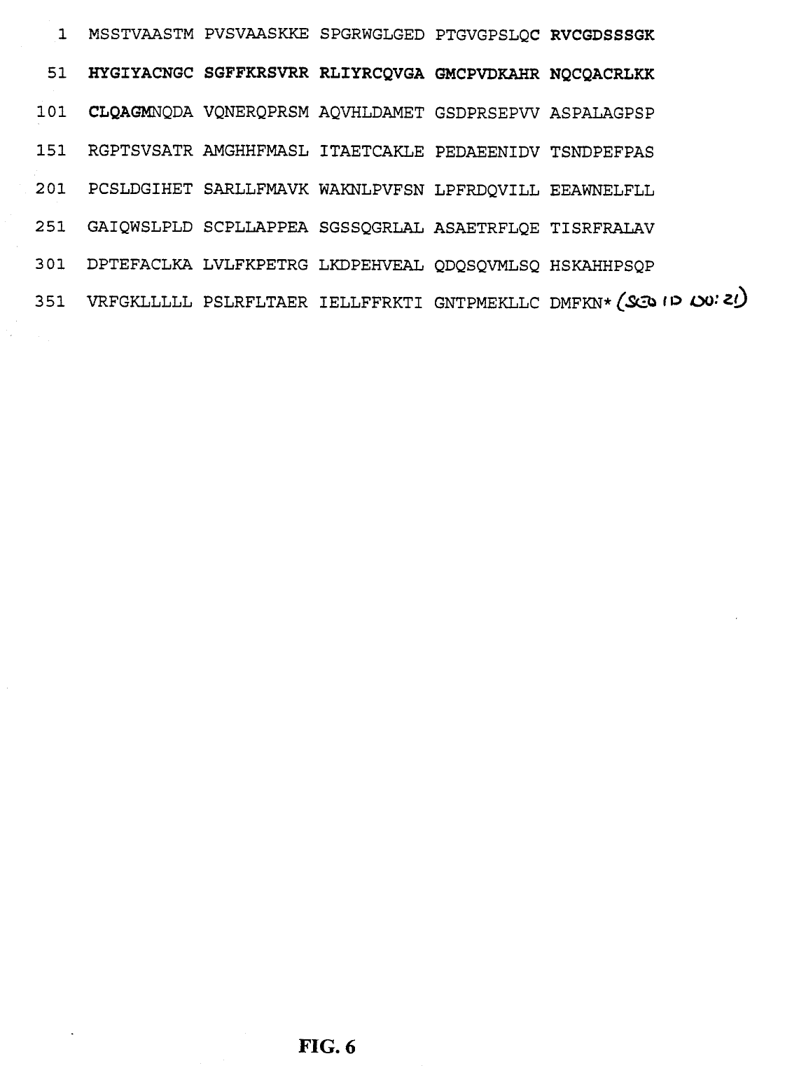

The present invention also relates to a substantially purified form of the novel mouse nuclear trans-acting receptor protein, nNR5, which is shown in FIGS. 5A-C and FIG. 6 and as set forth in SEQ ID NO:21, disclosed as follows:

(SEQ ID NO:21)
MSSTVAASTM PVSVAASKKE SPGRWGLGED PTGVGPSLQC
RVCGDSSSGK
HYGIYACNGC SGFFKRSVRR RLIYRCQVGA GMCPVDKAHR
NQCQACRLKK
CLQAGMNQDA VQNERQPRSM AQVHLDAMET GSDPRSEPVV
ASPALAGPSP
RGPTSVSATR AMGHHFMASL ITAETCAKLE PEDAEENIDV
TSNDPEFPAS
PCSLDGIHET SARLLFMAVK WAKNLPVFSN LPFRDQVILL
EEAWNELFLL
GAIQWSLPLD SCPLLAPPEA SGSSQGRLAL ASAETRFLQE
TISRFRALAV
DPTEFACLKA LVLFKPETRG LKDPEHVEAL QDQSQVMLSQ
HSKAHHPSQP
VRFGKLLLLL PSLRFLTAER IELLFFRKTI GNTPMEKLLC
DMFKN.

The region from Cys-39 to Met-106 represents the DNA binding domain (DBD) for this exemplified mouse nNR5 receptor protein. SEQ ID NO:21 contains 1623 nucleotides. This DNA molecule contains an open reading frame from nucleotide 58 (initiating Met from nt 58-60) to nucleotide 1242, with a “TAG” termination codon from nucleotides 1243-1245. This open reading frame encodes the 395 amino acid mouse nNR5 protein as set forth in SEQ ID NO:21. Again, due to its high expression in retinal tissue, nNR5 should play an important role in eye function. Therapeutic compounds may be selected which interact with and regulate nNR5 activity in retina tissue which may be involved with diseases of the eye, including but not limited to cataracts and glaucoma, as well as retina-specific diseases such as diabetes mellitus, retinitis pigmentosa, macular degeneration, retinal detachment and retinablastoma.

The present invention also relates to biologically functional derivatives and/or mutants of nNR5 as set forth as SEQ ID NO:2 and SEQ ID NO:21, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR5 function.

The present invention also relates to isolated nucleic acid molecules which are fusion constructions expressing fusion proteins useful in assays to identify compounds which modulate wild-type human and/or mouse nNR5 activity. A preferred aspect of this portion of the invention includes, but is not limited to, glutathione S-transferase GST-nNR5 fusion constructs. These fusion constructs include, but are not limited to, all or a portion of the ligand-binding domain of nNR5, respectively, as an in-frame fusion at the carboxy terminus of the GST gene. The disclosure of SEQ ID NOS:1, 2, 18, 19, 20 and 21 provide the artisan of ordinary skill the information necessary to construct any such nucleic acid molecule encoding a GST-nuclear receptor fusion protein. Soluble recombinant GST-nuclear receptor fusion proteins may be expressed in various expression systems, including Spodoptera frugiperda (Sf21) insect cells (Invitrogen) using a baculovirus expression vector (e.g., Bac-N-Blue DNA from Invitrogen or pAcG2T from Pharmingen).

Additionally, the present invention relates to constructs as disclosed in Example 3, wherein a receptor construct (containing the nNR5 LBD, e.g., Ga14-nNR5-LBD) and a reporter construct (such as SEAP or LacZ) with regulatory sites which respond to increases and decreases in expression of the receptor construct. Therefore, the present invention includes assays by which modulators of nNR5 are identified. Methods for identifying agonists and antagonists of other receptors are well known in the art and can be adapted to identify compounds which effect in vivo levels of nNR5. Accordingly, the present invention includes a method for determining whether a substance is a potential modulator of mouse or human nNR5 levels that comprises:

(a) transfecting or transforming cells with an expression vector encoding nNR5, (such as the LBD of nNR5) also known as the receptor vector;

(b) transfecting or transforming the cells of step (a) with second expression vector which comprises a response element known to bind nNR5 or a promoter fragment fused upstream of a reporter gene, also known as a reporter vector.

(c) allowing the transfected cells to grow for a time sufficient to allow nNR5 to be expressed;

(d) exposing the test cells to a substance while not exposing control cells to the test substance;

(e) measuring the expression of the reporter gene in both the test cells and control cells.

The conditions under which step (d) of the method is practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

Alternatively, the transactivation assay may be carried out as follows:

(a) provide test cells by transfecting cells with a receptor expression vector that directs the expression of nNR5 or a portion thereof (such as the LBD of nNR5) in the cells;

(b) providing test cells by transfecting the cells of step (a) with a second reporter expression vector that directs expression of a reporter gene under control of a regulatory element which is responsive to nNR5 or a portion of the nNR5 construct

(b) exposing the test cells to the substance;

(c) measuring the amount of binding of expression of the reporter gene;

(d) comparing the amount of expression of the reporter gene in the test cells with the amount of expression of the reporter gene in control cells that has been transfected with a reporter vector of step (b) but not a receptor vector of step (a).

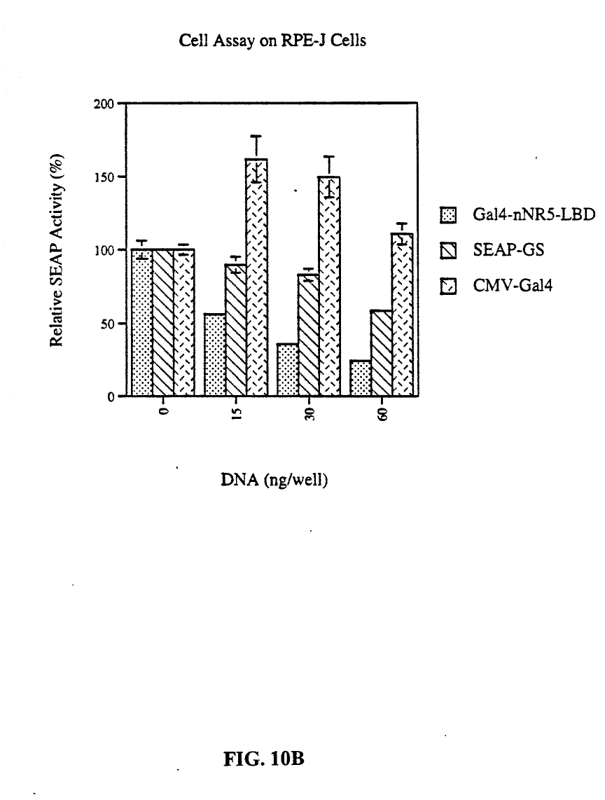

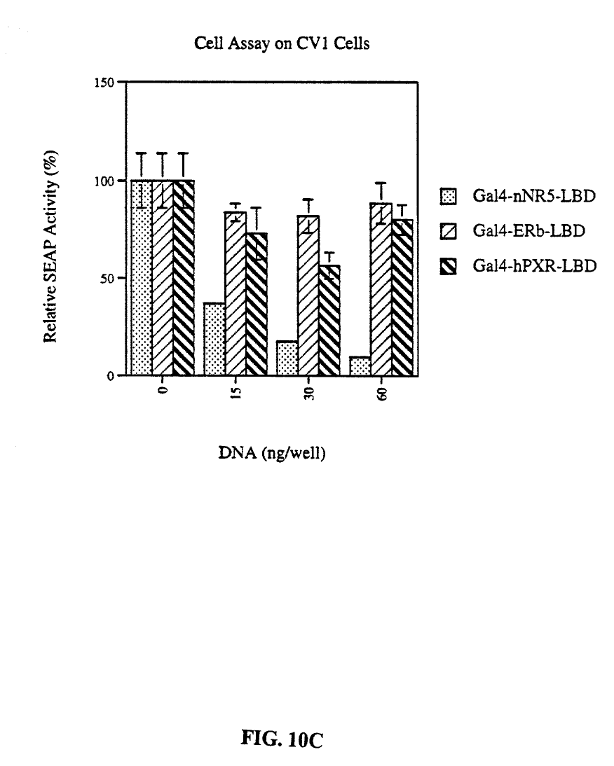

An alternative assay would be one such as is described in Example 3, wherein multiple receptor/reporter constructs are transfected into cells such the general nature of the trans-acting factor can be measured. In the case of human nNR5, Example 3 and FIGS. 10A-C show the inhibitory nature of the nNR5 trans-acting factor. Therefore, it is evident that any number of variations known to one of skill in the art may be utilized in order to provide for an assay to measure the effect of a substance on the ability of the nuclear receptor proteins of the present invention to effect transcription of a promoter of interest.

The present invention also includes a method for determining whether a substance is capable of binding to nNR5, i.e., whether the substance is a potential agonist or an antagonist of nNR5, where the method comprises:

(a) providing test cells by transfecting cells with an expression vector that directs the expression of nNR5 in the cells;

(b) exposing the test cells to the substance;

(c) measuring the amount of binding of the substance to nNR5;

(d) comparing the amount of binding of the substance to nNR5 in the test cells with the amount of binding of the substance to control cells that have not been transfected with nNR5 or a portion thereof; wherein if the amount of binding of the substance is greater in the test cells as compared to the control cells, the substance is capable of binding to nNR5. Determining whether the substance is actually an agonist or antagonist can then be accomplished by the use of functional assays such as the transactivation assay as described above.

The conditions under which step (b) of the method is practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

The assays described above can be carried out with cells that have been transiently or stably transfected with nNR5. Transfection is meant to include any method known in the art for introducing nNR5 into the test cells. For example, transfection includes calcium phosphate or calcium chloride mediated transfection, lipofection, infection with a retroviral construct containing nNR5, and electroporation.

Where binding of the substance or agonist to nNR5 is measured, such binding can be measured by employing a labeled substance or agonist. The substance or agonist can be labeled in any convenient manner known to the art, e.g., radioactively, fluorescently, enzymatically.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA). The preferred template is DNA.

It is known that there is a substantial amount of redundancy in the various codons which code for specific amino acids. Therefore, this invention is also directed to those DNA sequences encode RNA comprising alternative codons which code for the eventual translation of the identical amino acid, as shown below:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His =Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asp=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU.

Therefore, the present invention discloses codon redundancy which may result in differing DNA molecules expressing an identical protein. For purposes of this specification, a sequence bearing one or more replaced codons will be defined as a degenerate variation. Also included within the scope of this invention are mutations either in the DNA sequence or the translated protein which do not substantially alter the ultimate physical properties of the expressed protein. For example, substitution of valine for leucine, arginine for lysine, or asparagine for glutamine may not cause a change in functionality of the polypeptide.

It is known that DNA sequences coding for a peptide may be altered so as to code for a peptide having properties that are different than those of the naturally occurring peptide. Methods of altering the DNA sequences include but are not limited to site directed mutagenesis. Examples of altered properties include but are not limited to changes in the affinity of an enzyme for a substrate or a receptor for a ligand.

As used herein, “purified” and “isolated” are utilized interchangeably to stand for the proposition that the nucleic acid, protein, or respective fragment thereof in question has been substantially removed from its in vivo environment so that it may be manipulated by the skilled artisan, such as but not limited to nucleotide sequencing, restriction digestion, site-directed mutagenesis, and subcloning into expression vectors for a nucleic acid fragment as well as obtaining the protein or protein fragment in pure quantities so as to afford the opportunity to generate polyclonal antibodies, monoclonal antibodies, amino acid sequencing, and peptide digestion. Therefore, the nucleic acids claimed herein may be present in whole cells or in cell lysates or in a partially purified or substantially purified form. A nucleic acid is considered substantially purified when it is purified away from environmental contaminants. Thus, a nucleic acid sequence isolated from cells is considered to be substantially purified when purified from cellular components by standard methods while a chemically synthesized nucleic acid sequence is considered to be substantially purified when purified from its chemical precursors.

The present invention also relates to recombinant vectors and recombinant hosts, both prokaryotic and eukaryotic, which contain the substantially purified nucleic acid molecules disclosed throughout this specification.

Therefore, the present invention also relates to methods of expressing human and/or mouse nNR5 and biological equivalents disclosed herein, assays employing these recombinantly expressed gene products, cells expressing these gene products, and agonistic and/or antagonistic compounds identified through the use of assays utilizing these recombinant forms, including, but not limited to, one or more modulators of the human and/or mouse nNR5 either through direct contact LBD or through direct or indirect contact with a ligand which either interacts with the DBD or with the wild-type transcription complex which nNR5 interacts in trans, thereby modulating cell differentiation or cell development.

Therefore, the present invention relates to methods of expressing human or mouse nNR5 in recombinant systems and of identifying agonists and antagonists of nNR5. The novel nNR5 protein of the present invention is suitable for use in an assay procedure for the identification of compounds which modulate the transactivation activity of mammalian nNR5. Modulating nNR5 activity, as described herein includes the inhibition or activation of this soluble transacting factor and therefore includes directly or indirectly affecting the normal regulation of the nNR5 activity. Compounds which modulate nNR5 include agonists, antagonists and compounds which directly or indirectly affect regulation of human and/or mouse nNR5. When screening compounds in order to identify potential pharmaceuticals that specifically interact with a target protein, it is necessary to ensure that the compounds identified are as specific as possible for the target protein. To do this, it may necessary to screen the compounds against as wide an array as possible of proteins that are similar to the target receptor, including species homologous such as human and mouse nNR5. Thus, in order to find compounds that are potential pharmaceuticals that interact with nNR5, it is necessary not only to ensure that the compounds interact with nNR5 (the “plus target”) and produce the desired pharmacological effect through nNR5, it is also necessary to determine that the compounds do not interact with proteins B, C, D, etc. (the “minus targets”). In general, as part of a screening program, it is important to have as many minus targets as possible (see Hodgson, 1992, Bio/Technology 10:973-980, @980). Human and/or mouse nNR5 proteins and the DNA molecules encoding this protein have the additional utility in that they can be used as “minus targets” in screens designed to identify compounds that specifically interact with signal transduction within retinal tissue.

As used herein, a “biologically functional derivative” of a wild-type human nNR5 possesses a biological activity that is related to the biological activity of the wild type human or mouse nNR5. The term “functional derivative” is intended to include the “fragments,” “mutants,” “variants,” “degenerate variants,” “analogs” and “homologues” of the wild type human or mouse nNR5 protein. The term “fragment” is meant to refer to any polypeptide subset of wild-type human or mouse nNR5, including but not necessarily limited to nNR5 proteins comprising amino acid substitutions, deletions, additions, amino terminal truncations and/or carboxy-terminal truncations. The term “mutant” is meant to refer a subset of a biologically active fragment that may be substantially similar to the wild-type form but possesses distinguishing biological characteristics. Such altered characteristics include but are in no way limited to altered substrate binding, altered substrate affinity and altered sensitivity to chemical compounds affecting biological activity of the human or mouse nNR5 or human or mouse nNR5 functional derivative. The term “variant” is meant to refer to a molecule substantially similar in structure and function to either the entire wild-type protein or to a fragment thereof. A molecule is “substantially similar” to a wild-type human or mouse nNR5-like protein if both molecules have substantially similar structures or if both molecules possess similar biological activity. Therefore, if the two molecules possess substantially similar activity, they are considered to be variants even if the structure of one of the molecules is not found in the other or even if the two amino acid sequences are not identical. The term “analog” refers to a molecule substantially similar in function to either the full-length human or mouse nNR5 protein or to a biologically functional derivative thereof.

Any of a variety of procedures may be used to clone human or mouse nNR5. These methods include, but are not limited to, (1) a RACE PCR cloning technique (Frohman, et al., 1988, Proc. Natl. Acad. Sci. USA 85: 8998-9002). 5′ and/or 3′ RACE may be performed to generate a full length cDNA sequence. This strategy involves using gene-specific oligonucleotide primers for PCR amplification of human or mouse nNR5 cDNA. These gene-specific primers are designed through identification of an expressed sequence tag (EST) nucleotide sequence which has been identified by searching any number of publicly available nucleic acid and protein databases; (2) direct functional expression of the human or mouse nNR5 cDNA following the construction of a human or mouse nNR5-containing cDNA library in an appropriate expression vector system; (3) screening a human or mouse nNR5-containing cDNA library constructed in a bacteriophage or plasmid shuttle vector with a labeled degenerate oligonucleotide probe designed from the amino acid sequence of the human or mouse nNR5 protein; (4) screening a human or mouse nNR5-containing cDNA library constructed in a bacteriophage or plasmid shuttle vector with a partial cDNA encoding the human or mouse nNR5 protein. This partial cDNA is obtained by the specific PCR amplification of human or mouse nNR5 DNA fragments through the design of degenerate oligonucleotide primers from the amino acid sequence known for other kinases which are related to the human or mouse nNR5 protein; (5) screening a human or mouse nNR5-containing cDNA library constructed in a bacteriophage or plasmid shuttle vector with a partial cDNA encoding the human or mouse nNR5 protein. This strategy may also involve using gene-specific oligonucleotide primers for PCR amplification of human or mouse nNR5 cDNA identified as an EST as described above; or (6) designing 5′ and 3′ gene specific oligonucleotides using SEQ ID NO:1, 18, 19 or 20 as a template so that either the full-length cDNA may be generated by known PCR techniques, or a portion of the coding region may be generated by these same known PCR techniques to generate and isolate a portion of the coding region to use as a probe to screen one of numerous types of cDNA and/or genomic libraries in order to isolate a full-length version of the nucleotide molecule encoding human or mouse nNR5.

It is readily apparent to those skilled in the art that other types of libraries, as well as libraries constructed from other cell types-or species types, may be useful for isolating a nNR5-encoding DNA or a nNR5 homologue. Other types of libraries include, but are not limited to, cDNA libraries derived from other cells or cell lines other than human cells or tissue such as murine cells, rodent cells or any other such vertebrate host which may contain nNR5-encoding DNA. Additionally a nNR5 gene and homologues may be isolated by oligonucleotide- or polynucleotide-based hybridization screening of a vertebrate genomic library, including but not limited to, a murine genomic library, a rodent genomic library, as well as concomitant human genomic DNA libraries.

It is readily apparent to those skilled in the art that suitable cDNA libraries may be prepared from cells or cell lines which have nNR5 activity. The selection of cells or cell lines for use in preparing a cDNA library to isolate a cDNA encoding nNR5 may be done by first measuring cell-associated nNR5 activity using any known assay available for such a purpose.

Preparation of cDNA libraries can be performed by standard techniques well known in the art. Well known cDNA library construction techniques can be found for example, in Sambrook et al., 1989, Molecular Cloning: A Laboratory Manual; Cold Spring Harbor Laboratory, Cold Spring Harbor, N.Y. Complementary DNA libraries may also be obtained from numerous commercial sources, including but not limited to Clontech Laboratories, Inc. and Stratagene.

It is also readily apparent to those skilled in the art that DNA encoding human or mouse nNR5 may also be isolated from a suitable genomic DNA library. Construction of genomic DNA libraries can be performed by standard techniques well known in the art. Well known genomic DNA library construction techniques can be found in Sambrook, et al., supra.

In order to clone the human or mouse nNR5 gene by one of the preferred methods, the amino acid sequence or DNA sequence of human or mouse nNR5 or a homologous protein may be necessary. To accomplish this, the nNR5 protein or a homologous protein may be purified and partial amino acid sequence determined by automated sequenators. It is not necessary to determine the entire amino acid sequence, but the linear sequence of two regions of 6 to 8 amino acids can be determined for the PCR amplification of a partial human or mouse nNR5 DNA fragment. Once suitable amino acid sequences have been identified, the DNA molecules capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the human or mouse nNR5 sequence but others in the set will be capable of hybridizing to human or mouse nNR5 DNA even in the presence of DNA oligonucleotides with mismatches. The mismatched DNA oligonucleotides may still sufficiently hybridize to the human or mouse nNR5 DNA to permit identification and isolation of human or mouse nNR5 encoding DNA. Alternatively, the nucleotide sequence of a region of an expressed sequence may be identified by searching one or more available genomic databases. Gene-specific primers may be used to perform PCR amplification of a cDNA of interest from either a cDNA library or a population of cDNAs. As noted above, the appropriate nucleotide sequence for use in a PCR-based method may be obtained from SEQ ID NO: 1 or 18-20, either for the purpose of isolating overlapping 5′ and 3′ RACE products for generation of a full-length sequence coding for human or mouse nNR5, or to isolate a portion of the nucleotide molecule coding for human or mouse nNR5 for use as a probe to screen one or more cDNA- or genomic-based libraries to isolate a full-length molecule encoding human or mouse nNR5 or human or mouse nNR5-like proteins.

In an exemplified method, the human nNR5 full-length cDNA of the present invention was isolated by screening a human retina cDNA library with an oligonucleotide primer pair to a human EST identified herein as SEQ ID NO: 3. Positive cDNA clones were sequenced and shown to possess an intron. This cDNA was subjected to sequence analysis and is reported herein and is set forth as SEQ ID NO: 18. A second oligonucleotide primer pair which flanks the putative intron was used to rescreen the human retina cDNA library. Shorter cDNA clones (about 2.1 kb) were chosen for sequence analysis and shown to comprise an uninterrupted open reading frame (e.g., SEQ ID NO:1) encoding human nNR5 (SEQ ID NO: 2). The intron-containing clone disclosed as SEQ ID NO: 18 contains 70 additional nucleotides at the 5′ end of the cDNA clone. Therefore, an additional isolated DNA molecule of the present invention includes but is not limited to the DNA molecule as set forth herein and as set forth as SEQ ID NO: 19.

A variety of mammalian expression vectors may be used to express recombinant human or mouse nNR5 in mammalian cells. Expression vectors are defined herein as DNA sequences that are required for the transcription of cloned DNA and the translation of their mRNAs in an appropriate host. Such vectors can be used to express eukaryotic DNA in a variety of hosts such as bacteria, blue green algae, plant cells, insect cells and animal cells. Specifically designed vectors allow the shuttling of DNA between hosts such as bacteria-yeast or bacteria-animal cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses.

Commercially available mammalian expression vectors which may be suitable for recombinant human nNR5 expression, include but are not limited to, pcDNA3.1 (Invitrogen), pLITMUS28, pLITMUS29, pLITMUS38 and pLITMUS39 (New England Bioloabs), pcDNAI, pcDNAlamp (Invitrogen), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565).

A variety of bacterial expression vectors may be used to express recombinant human or mouse nNR5 in bacterial cells. Commercially available bacterial expression vectors which may be suitable for recombinant human nNR5 expression include, but are not limited to pCRII (Invitrogen), pCR2.1 (Invitrogen), pQE (Qiagen), pET11a (Novagen), lambda gt11 (Invitrogen), and pKK223-3 (Pharmacia).

A variety of fungal cell expression vectors may be used to express recombinant human or mouse nNR5 in fungal cells. Commercially available fungal cell expression vectors which may be suitable for recombinant human or mouse nNR5 expression include but are not limited to pYES2 (Invitrogen) and Pichia expression vector (Invitrogen).

A variety of insect cell expression vectors may be used to express recombinant receptor in insect cells. Commercially available insect cell expression vectors which may be suitable for recombinant expression of human or mouse nNR5 include but are not limited to pBlueBacIII and pBlueBacHis2 (Invitrogen), and pAcG2T (Pharmingen).

An expression vector containing DNA encoding a human or mouse nNR5-like protein may be used for expression of human or mouse nNR5 in a recombinant host cell. Recombinant host cells may be prokaryotic or eukaryotic, including but not limited to bacteria such as E. coli, fungal cells such as yeast, mammalian cells including but not limited to cell lines of human, bovine, porcine, monkey and rodent origin, and insect cells including but not limited to Drosophila- and silkworm-derived cell lines. Cell lines derived from mammalian species which may be suitable and which are commercially available, include but are not limited to, L cells L-M(TK⁻) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

The expression vector may be introduced into host cells via any one of a number of techniques including but not limited to transformation, transfection, protoplast fusion, and electroporation. The expression vector-containing cells are individually analyzed to determine whether they produce human or mouse nNR5 protein. Identification of human or mouse nNR5 expressing cells may be done by several means, including but not limited to immunological reactivity with anti-human or anti-mouse nNR5 antibodies, labeled ligand binding and the presence of host cell-associated human or mouse nNR5 activity.

The cloned human or mouse nNR5 cDNA obtained through the methods described above may be recombinantly expressed by molecular cloning into an expression vector (such as pcDNA3.1, pQE, pBlueBacHis2 and pLITMUS28) containing a suitable promoter and other appropriate transcription regulatory elements, and transferred into prokaryotic or eukaryotic host cells to produce recombinant human or mouse nNR5. Techniques for such manipulations can be found described in Sambrook, et al., supra, are discussed at length in the Example section and are well known and easily available to the artisan of ordinary skill in the art.

Expression of human or mouse nNR5 DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

To determine the human or mouse nNR5 cDNA sequence(s) that yields optimal levels of human or mouse nNR5, cDNA molecules including but not limited to the following can be constructed (describing a human cDNA, but also applying to other mammalian nNR5 cDNA, such as mouse): a cDNA fragment containing the full-length open reading frame for human nNR5 as well as various constructs containing portions of the cDNA encoding only specific domains of the protein or rearranged domains of the protein. All constructs can be designed to contain none, all or portions of the 5′ and/or 3′ untranslated region of a human nNR5 cDNA. The expression levels and activity of human nNR5 can be determined following the introduction, both singly and in combination, of these constructs into appropriate host cells. Following determination of the human nNR5 cDNA cassette yielding optimal expression in transient assays, this nNR5 cDNA construct is transferred to a variety of expression vectors (including recombinant viruses), including but not limited to those for mammalian cells, plant cells, insect cells, oocytes, bacteria, and yeast cells.

The present invention also relates to a non-human transgenic animal which is heterozygous for a functional nNR5 gene native to that animal. As used herein, functional is used to describe a gene or protein that, when present in a cell or in vitro system, performs normally as if in a native or unaltered condition or environment. The animal of this aspect of the invention is useful for the study of the retinal specific expression or activity of nNR5 in an animal having only one functional copy of the gene. The animal is also useful for studying the ability of a variety of compounds to act as modulators of nNR5 activity or expression in vivo or, by providing cells for culture, in vitro. It is reiterated that as used herein, a modulator is a compound that causes a change in the expression or activity of nNR5, or causes a change in the effect of the interaction of nNR5 with its ligand(s), or other protein(s). In an embodiment of this aspect, the animal is used in a method for the preparation of a further animal which lacks a functional native nNR5 gene. In another embodiment, the animal of this aspect is used in a method to prepare an animal which expresses a non-native nNR5 gene in the absence of the expression of a native nNR5 gene. In particular embodiments the non-human animal is a mouse. In further embodiments the non-native nNR5 is a wild-type human nNR5 which is disclosed herein, or any other biologically equivalent form of human nNR5 gene as also disclosed herein.

In reference to the transgenic animals of this invention, reference is made to transgenes and genes. As used herein, a transgene is a genetic construct including a gene. The transgene is integrated into one or more chromosomes in the cells in an animal by methods known in the art. Once integrated, the transgene is carried in at least one place in the chromosomes of a transgenic animal. Of course, a gene is a nucleotide sequence that encodes a protein, such as human or mouse nNR5. The gene and/or transgene may also include genetic regulatory elements and/or structural elements known in the art.

Another aspect of the invention is a non-human animal embryo deficient for native nNR5 expression. This embryo is useful in studying the effects of the lack of nNR5 on the developing animal. In particular embodiments the animal is a mouse. The animal embryo is also useful as a source of cells lacking a functional native nNR5 gene. The cells are useful in in vitro culture studies in the absence of nNR5.

An aspect of this invention is a method to obtain an animal in which the cells lack a functional gene nNR5 native to the animal. The method includes providing a gene for an altered form of the nNR5 gene native to the animal in the form of a transgene and targeting the transgene into a chromosome of the animal at the place of the native nNR5 gene. The transgene can be introduced into the embryonic stem cells by a variety of methods known in the art, including electroporation, microinjection, and lipofection. Cells carrying the transgene can then be injected into blastocysts which are then implanted into pseudopregnant animals. In alternate embodiments, the transgene-targeted embryonic stem cells can be coincubated with fertilized eggs or morulae followed by implantation into females. After gestation, the animals obtained are chimeric founder transgenic animals. The founder animals can be used in further embodiments to cross with wild-type animals to produce F1 animals heterozygous for the altered nNR5 gene. In further embodiments, these heterozygous animals can be interbred to obtain the non-viable transgenic embryos whose somatic and germ cells are homozygous for the altered nNR5 gene and thereby lack a functional nNR5 gene. In other embodiments, the heterozygous animals can be used to produce cells lines. In preferred embodiments, the animals are mice.

A further aspect of the present invention is a transgenic non-human animal which expresses a non-native nNR5 on a native nNR5 null background. In particular embodiments, the null background is generated by producing an animal with an altered native nNR5 gene that is non-functional, i.e. a knockout. The animal can be heterozygous (i.e., having a different allelic representation of a gene on each of a pair of chromosomes of a diploid genome) or homozygous (i.e., having the same representation of a gene on each of a pair of chromosomes of a diploid genome) for the altered nNR5 gene and can be hemizygous (i.e., having a gene represented on only one of a pair of chromosomes of a diploid genome) or homozygous for the non-native nNR5 gene. In preferred embodiments, the animal is a mouse. In particular embodiments the non-native nNR5 gene can be a wild-type or mutant allele including those mutant alleles associated with a disease. In further embodiments, the non-native nNR5 is a human nNR5. In a further embodiment the non-native nNR5 gene is operably linked to a promoter. As used herein, operably linked is used to denote a functional connection between two elements whose orientation relevant to one another can vary. In this particular case, it is understood in the art that a promoter can be operably linked to the coding sequence of a gene to direct the expression of the coding sequence while placed at various distances from the coding sequence in a genetic construct.

An aspect of this invention is a method of producing transgenic animals having a transgene including a non-native nNR5 gene on a native nNR5 null background. The method includes providing transgenic animals of this invention whose cells are heterozygous for a native gene encoding a functional nNR5 protein and an altered native nNR5 gene. These animals are crossed with transgenic animals of this invention that are hemizygous for a transgene including a non-native nNR5 gene to obtain animals that are both heterozygous for an altered native nNR5 gene and hemizygous for a non-native nNR5 gene. The latter animals are interbred to obtain animals that are homozygous or hemizygous for the non-native nNR5 and are homozygous for the altered native nNR5 gene. In particular embodiments, cell lines are produced from any of the animals produced in the steps of the method.

The transgenic animals and cells of this invention are useful in the determination of the in vivo function of a non-native nNR5 in the central nervous system and in other tissues of an animal. The animals are also useful in studying the tissue and temporal specific expression patterns of a non-native nNR5 throughout the animals. The animals are also useful in determining the ability for various forms of wild-type and mutant alleles of a non-native nNR5 to rescue the native nNR5 null deficiency. The animals are also useful for identifying and studying the ability of a variety of compounds to act as modulators of the expression or activity of a non-native nNR5 in vivo, or by providing cells for culture, for in vitro studies.

As used herein, a “targeted gene” or “Knockout” (KO) is a DNA sequence introduced into the germline of a non-human animal by way of human intervention, including but not limited to, the methods described herein. The targeted genes of the invention include nucleic acid sequences which are designed to specifically alter cognate endogenous alleles. An altered nNR5 gene should not fully encode the same nNR5 as native to the host animal, and its expression product can be altered to a minor or great degree, or absent altogether. In cases where it is useful to express a non-native nNR5 gene in a transgenic animal in the absence of a native nNR5 gene we prefer that the altered nNR5 gene induce a null lethal knockout phenotype in the animal. However a more modestly modified nNR5 gene can also be useful and is within the scope of the present invention.

A type of target cell for transgene introduction is the embryonic stem cell (ES). ES cells can be obtained from pre-implantation embryos cultured in vitro and fused with embryos (Evans et al., 1981, Nature 292:154-156; Bradley et al., 1984, Nature 309:255-258; Gossler et al., 1986, Proc. Natl. Acad. Sci. USA 83:9065-9069; and Robertson et al., 1986 Nature 322:445-448). Transgenes can be efficiently introduced into the ES cells by a variety of standard techniques such as DNA transfection, microinjection, or by retrovirus-mediated transduction. The resultant transformed ES cells can thereafter be combined with blastocysts from a non-human animal. The introduced ES cells thereafter colonize the embryo and contribute to the germ line of the resulting chimeric animal (Jaenisch, 1988, Science 240: 1468-1474).

The methods for evaluating the targeted recombination events as well as the resulting knockout mice are readily available and known in the art. Such methods include, but are not limited to DNA (Southern) hybridization to detect the targeted allele, polymerase chain reaction (PCR), polyacrylamide gel electrophoresis (PAGE) and Western blots to detect DNA, RNA and protein.

The present invention also relates to polyclonal and monoclonal antibodies raised in response to either the human or mouse form of nNR5 disclosed herein, or a biologically functional derivative thereof. It will be especially preferable to raise antibodies against epitopes within the NH₂-terminal domain of nNR5, which show the least homology to other known proteins belonging to the human nuclear receptor superfamily.

Recombinant nNR5 protein can be separated from other cellular proteins by use of an immunoaffinity column made with monoclonal or polyclonal antibodies specific for full-length nNR5 protein, or polypeptide fragments of nNR5 protein. Additionally, polyclonal or monoclonal antibodies may be raised against a synthetic peptide (usually from about 9 to about 25 amino acids in length) from a portion of the protein as disclosed in SEQ ID NO:2 and/or SEQ ID NO:21. Monospecific antibodies to human or mouse nNR5 are purified from mammalian antisera containing antibodies reactive against human or mouse nNR5 or are prepared as monoclonal antibodies reactive with human nNR5 using the technique of Kohler and Milstein (1975, Nature 256: 495-497). Monospecific antibody as used herein is defined as a single antibody species or multiple antibody species with homogenous binding characteristics for human nNR5. Homogenous binding as used herein refers to the ability of the antibody species to bind to a specific antigen or epitope, such as those associated with human or mouse nNR5, as described above. Human or mouse nNR5-specific antibodies are raised by immunizing animals such as mice, rats, guinea pigs, rabbits, goats, horses and the like, with an appropriate concentration of human or mouse nNR5 protein or a synthetic peptide generated from a portion of human or mouse nNR5 with or without an immune adjuvant.

Preimmune serum is collected prior to the first immunization. Each animal receives between about 0.1 mg and about 1000 mg of human or mouse nNR5 protein associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of human or mouse nNR5 protein or peptide fragment thereof in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of human or mouse nNR5 in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with human or mouse nNR5 are prepared by immunizing inbred mice, preferably Balb/c, with human or mouse nNR5 protein. The mice are immunized by the IP or SC route with about 1 mg to about 100 mg, preferably about 10 mg, of human or mouse nNR5 protein in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 1 to about 100 mg of human or mouse nNR5 in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1, MPC-11, S-194 and Sp 2/0, with Sp 2/0 being preferred. The antibody producing cells and myeloma cells are fused in polyethylene glycol, about 1000 mol. wt., at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected form growth positive wells on about days 14, 18, and 21 and are screened for antibody production by an immunoassay such as solid phase immunoradioassay (SPIRA) using human or mouse nNR5 as the antigen. The culture fluids are also tested in the Ouchterlony precipitation assay to determine the isotype of the mAb. Hybridoma cells from antibody positive wells are cloned by a technique such as the soft agar technique of MacPherson, 1973, Soft Agar Techniques, in Tissue Culture Methods and Applications, Kruse and Paterson, Eds., Academic Press.

Monoclonal antibodies are produced in vivo by injection of pristine primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of anti-human or mouse nNR5 mAb is carried out by growing the hybridoma in DMEM containing about 2% fetal calf serum to obtain sufficient quantities of the specific mAb. The mAb are purified by techniques known in the art.

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. Similar assays are used to detect the presence of human nNR5 in body fluids or tissue and cell extracts.

It is readily apparent to those skilled in the art that the above described methods for producing monospecific antibodies may be utilized to produce antibodies specific for human or mouse nNR5 peptide fragments, or full-length human nNR5.

Human or mouse nNR5 antibody affinity columns are made, for example, by adding the antibodies to Affigel-10 (Biorad), a gel support which is pre-activated with N-hydroxysuccinimide esters such that the antibodies form covalent linkages with the agarose gel bead support. The antibodies are then coupled to the gel via amide bonds with the spacer arm. The remaining activated esters are then quenched with 1M ethanolamine HCl (pH 8.0). The column is washed with water followed by 0.23 M glycine HCl (pH 2.6) to remove any non-conjugated antibody or extraneous protein. The column is then equilibrated in phosphate buffered saline (pH 7.3) and the cell culture supernatants or cell extracts containing full-length human nNR5 or human nNR5 protein fragments are slowly passed through the column. The column is then washed with phosphate buffered saline until the optical density (A280) falls to background, then the protein is eluted with 0.23 M glycine-HCl (pH 2.6). The purified human or mouse nNR5 protein is then dialyzed against phosphate buffered saline.

Levels of human or mouse nNR5 in host cells is quantified by a variety of techniques including, but not limited to, immunoaffinity and/or ligand affinity techniques. nNR5-specific affinity beads or nNR5-specific antibodies are used to isolate ³⁵S-methionine labeled or unlabelled nNR5. Labeled nNR5 protein is analyzed by SDS-PAGE. Unlabelled nNR5 protein is detected by Western blotting, ELISA or RIA assays employing either nNR5 protein specific antibodies and/or antiphosphotyrosine antibodies.

Following expression of nNR5 in a host cell, nNR5 protein may be recovered to provide nNR5 protein in active form. Several nNR5 protein purification procedures are available and suitable for use. Recombinant nNR5 protein may be purified from cell lysates and extracts, or from conditioned culture medium, by various combinations of, or individual application of salt fractionation, ion exchange chromatography, size exclusion chromatography, hydroxylapatite adsorption chromatography and hydrophobic interaction chromatography.

The present invention is also directed to methods for screening for compounds which modulate the expression of DNA or RNA encoding a human and/or mouse nNR5 protein. Compounds which modulate these activities may be DNA, RNA, peptides, proteins, or non-proteinaceous organic molecules. Compounds may modulate by increasing or attenuating the expression of DNA or RNA encoding human and/or mouse nNR5, or the function of human and/or mouse nNR5. Compounds that modulate the expression of DNA or RNA encoding human and/or mouse nNR5 or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample. Kits containing human or mouse nNR5, antibodies to human or mouse nNR5, or modified human or mouse nNR5 may be prepared by known methods for such uses. As noted earlier, the present invention relates to methods of expressing human or mouse nNR5 in recombinant systems and of identifying agonists and antagonists of nNR5. The novel nNR5 protein of the present invention is suitable for use in an assay procedure for the identification of compounds which modulate the transactivation activity of mammalian nNR5. Modulating nNR5 activity, as described herein includes the inhibition or activation of this soluble transacting factor and therefore includes directly or indirectly affecting the normal regulation of the nNR5 activity. Compounds which modulate nNR5 include agonists, antagonists and compounds which directly or indirectly affect regulation of human and/or mouse nNR5. When screening compounds in order to identify potential pharmaceuticals that specifically interact with a target protein, it is necessary to ensure that the compounds identified are as specific as possible for the target protein. To do this, it may necessary to screen the compounds against as wide an array as possible of proteins that are similar to the target receptor, including species homologous such as human and mouse nNR5. Thus, in order to find compounds that are potential pharmaceuticals that interact with nNR5, it is necessary not only to ensure that the compounds interact with nNR5 (the “plus target”) and produce the desired pharmacological effect through nNR5, it is also necessary to determine that the compounds do not interact with proteins B, C, D, etc. (the “minus targets”). In general, as part of a screening program, it is important to have as many minus targets as possible (see Hodgson, 1992, Bio/Technology 10:973-980, @980). Human and/or mouse nNR5 proteins and the DNA molecules encoding this protein have the additional utility in that they can be used as “minus targets” in screens designed to identify compounds that specifically interact with signal transduction within retinal tissue.

The DNA molecules, RNA molecules, recombinant protein and antibodies of the present invention may be used to screen and measure levels of human or mouse nNR5. The recombinant proteins, DNA molecules, RNA molecules and antibodies lend themselves to the formulation of kits suitable for the detection and typing of human or mouse nNR5. Such a kit would comprise a compartmentalized carrier suitable to hold in close confinement at least one container. The carrier would further comprise reagents such as recombinant nNR5 or anti-nNR5 antibodies suitable for detecting human or mouse nNR5. The carrier may also contain a means for detection such as labeled antigen or enzyme substrates or the like.

Pharmaceutically useful compositions comprising modulators of human or mouse nNR5 may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, modified human or mouse nNR5, or either nNR5 agonists or antagonists.

Therapeutic or diagnostic compositions comprising modulators of nNR5 are administered to an individual in amounts sufficient to treat or diagnose disorders. The effective amount may vary according to a variety of factors such as the individual condition, weight, sex and age. Other factors include the mode of administration.

The pharmaceutical compositions may be provided to the individual by a variety of routes such as subcutaneous, topical, oral and intramuscular.

The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Compounds identified according to the methods disclosed herein may be used alone at appropriate dosages. Alternatively, co-administration or sequential administration of other agents may be desirable.

The present invention also has the objective of providing suitable topical, oral, systemic and parenteral pharmaceutical formulations for use in the novel methods of treatment of the present invention. The compositions containing compounds identified according to this invention as the active ingredient can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds of the present invention is selected in accordance with a variety of factors including type, species, age, weight, sex and medical condition of the patient; the severity of the condition to be treated; the route of administration; the renal, hepatic and cardiovascular function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drugs availability to target sites. This involves a consideration of the distribution, equilibrium, and elimination of a drug.

The following examples are provided to illustrate the present invention without, however, limiting the same hereto.

### EXAMPLE 1

### Isolation and Characterization of a DNA Molecule Encoding nNR5

An EST from a human retina cDNA library was identified during a data base search. This EST is identified by GenBank Accession No. W27871 and dbEST Id No. 534939 and is disclosed as follows:

(SEQ ID NO:3)

| | |
|---|---|
| 1 | GGAATCACCA GGGGAGACAG GNGCACAGNG AGACAGAGGT |

TCATGGACTG

| | |
|---|---|
| 51 | AGGCAAAGGC TGGGCCAGGC TCAGCAACCC AGGCCTCCCG |

CAGGCAGGCA

| | |
|---|---|
| 101 | GAGGCTGCCC TGTAACCCAT GGAGACCAGA CCAACAGCTC |

TGATGAGCTC

| | |
|---|---|
| 151 | CACAGTGGCT GCAGCTGCGC CTGCAGCTGG GGCTGCCTCC |

AGGAAGGAGT

| | |
|---|---|
| 201 | CTCCAGGCAG ATGGGGCCTG GGGGAGGATC CCACAGGCGT |

GAGCCCCTCG

| | |
|---|---|
| 251 | CTCCAGTGCC GCGTGTGCGG AGACAGCAGC AGCGGGAAGC |

ACTATGGCAT

| | |
|---|---|
| 301 | CTATGCCCTG CAACGGTTGC AGCGGTTTCT TCCAAGAGGA |

GCNGTACGGN

| | |
|---|---|
| 351 | GGAGGCTCAA TCCTTACAAG GGTGCCCAGG GTGGGGGCAG |

GGATTGTGCC

| | |
|---|---|
| 401 | CCCCNGTGGA CAAGGNCCCA ACCCGNAACC CAGTGCCCAG |

GCCTGCCGGN

| | |
|---|---|
| 451 | TTGAGAAGTG CTTNAAAANN NGGNNGGGGN TTGAACCCAG |

GACGCCCGTN

| | |
|---|---|
| 501 | NAAAGGAACG ANNGCCNAGC CCGNGAGGAN AAGCCCAGGT |

NCCACCCCTG

| | |
|---|---|
| 551 | GANAAGAATN NNNNNNNNNN NNNNNNNNNN NNNNNNNNNN |

NNNNNNNNNN

| | |
|---|---|
| 601 | NNNNNNNNNN NNNNNNNNNN NNNNNNNNNN NNNNNNNNNN |

NNNNNNNNNN

| | |
|---|---|
| 651 | NNNNNNNNNN NNNNNNNNNN NNNNNNNNNN NNNMNNNNNN |

NNNNNNNNNN

| | |
|---|---|
| 701 | NNNNNNNNNN NNNNNNNNNN NNNNNNNNNN NNNNNNNNNN |

NNNNNNNNNN

| | |
|---|---|
| 751 | NNNNNNNNNN NNNNNNNNNN NNNNNNNNNN NNNNNNNNNN |

NNNNNNNNNN

| | |
|---|---|
| 801 | NNNNNNNNNN NNNNNNNNNN NNNNNNNNNN NNNNNNNNNN |

NNNNNNNNNN

| | |
|---|---|
| 851 | NNNNNNNNN. |

DNA fragments encoding DBD regions of androgen receptor (AR), estrogen receptor b (ERb), glucocorticoid receptor (GR) and vitamin D receptor (VDR) were generated by PCR and subcloned into pCR cloning vectors as described by the manufacturer. The following oligonucleotide primers were utilized to generate fragments for plasmid subcloning:

1. GR-R 5′-TTTCGAGCTTCCAGGTTCAT-3′ (SEQ ID NO: 6),

2. GR-F 5′-CTCCCAAACTCTGCCTGGTG-3′ (SEQ ID NO: 7),

3. ERB-R 5′-CGGGAGCCACACTTCACCAT-3′ (SEQ ID NO: 8),

4. ERB-F 5′-GCTCACTTCTGCGCTGTCTG-3′ (SEQ ID NO: 9),

5. AR-R 5′-TTCCGGGCTCCCAGAGTCAT-3′ (SEQ ID NO: 10),

6. AR-F 5′-CAGAAGACCTGCCTGATCTG-3′ (SEQ ID NO: 1),

7. VDR-R 5′-GAAATGAACTCCTTCATCAT-3′ (SEQ ID NO: 12),

8. VDR-F 5′-CCGGATCTGTGGGGTGTGTG-3′ (SEQ ID NO: 13).

PCR templates for AR, ERb and GR are cDNAs made from human fetal brain mRNA. PCR template for VDR was a cDNA made from human small intestine mRNA. The DNA fragments were purified using a Qiagen gel extraction kit. Phosphorylation, self-ligation and transformation of the purified DNA was carried out as recommended by the manufacturer. A human retina cDNA library was screened at low stringency using the above-identified AR, Erb, GR and VDR's DBD regions as probes. Two positive clones were selected and subjected to sequence analysis, which revealed the presence of an intron as shown herein and as set forth as SEQ ID NO: 18. Direct sequencing of plasmid DNA from clone A8 and A9 revealed a full cDNA molecule 3,012 bps in length (SEQ ID NO: 18), which encodes a peptide most related to hCOUP-TF (Wang et al., 1989, Nature 340: 163-166). These cDNA clones showed homology to the human EST (GenBank Accession No. W27871 and dbEST Id No. 534939; SEQ ID NO: 3).

To isolate an intronless cDNA clone for nNR5, the human retina cDNA library was screened by PCR analysis with primer pair nNR5F2 (5′-ATGAGCTCCACAGTGGCTGC-3′; SEQ ID NO: 4) and nNR5R (5′-CTGTCTCCGCACACGCGGCA-3′; SEQ ID NO: 5) from the human EST (GenBank Accession No. W27871 and dbEST Id No. 534939; SEQ ID NO:3). Further screening of the retina cDNA library by PCR using nNR5F2/nNR5R on retina cDNA resulted in a total of 20 positive clones from approximately 250,000 primary clones. This data indicated that the gene of interest (eventually identified as a cDNA encoding human nNR5) is abundantly expressed in retina tissue. In order to define the exact intron-exon boundary and to isolate an intronless cDNA, primer pair R5F3 (5′-CTGATGAGAATATTGATGT-3′; SEQ ID NO: 14) and R5R4 (5′-CGTGAGCCGGCCCTGGGCA-3′; SEQ ID NO: 15), which flank the putative intron region, was used in PCR on the twenty positive clones. Two clones, E1 and F6, yielded a band of smaller size than that of the A8 which had an intron. DNA fragments from this PCR were purified and submitted for sequencing. Automated sequencing was performed on and sequence assembly and analysis were performed with SEQUENCHER™ 3.0 (Gene Codes Corporation, Ann Arbor, Mich.). Ambiguities and/or discrepancies between automated base calling in sequencing reads were visually examined and edited to the correct base call. Based on the sequencing result and protein sequence alignment an intron region in the original A8/A9 clone was identified from nucleotide 971 to 1847. Therefore, the full length cDNA without an intron is approximately 2.1 kb and this DNA molecule which encodes human nNR5 is shown in FIGS. 1A-B and is set forth as SEQ ID NO:1.

In order to identify the genome map position of nNR5, primers in the 3′ non-coding region were designed. Forward primer R5F9 (5′-GGCATGGACCTCACTGAAGA-3′; SEQ ID NO: 16) and reverse primer R5R10 (5′-ACTGGCAGGAACCTGTTATA-3′; SEQ ID NO: 17) were used in PCR scanning on the 83 clones of the Stanford radiation hybrid panel (Cox et al., 1990, Science, 250:245-250). The PCR results were scored and submitted to the Stanford Genome Center for linkage analysis. The result indicates that nNR5 is located on chromosome 15.

### EXAMPLE 2

### Isolation and Characterization of a DNA Molecule Encoding Mouse nNR5

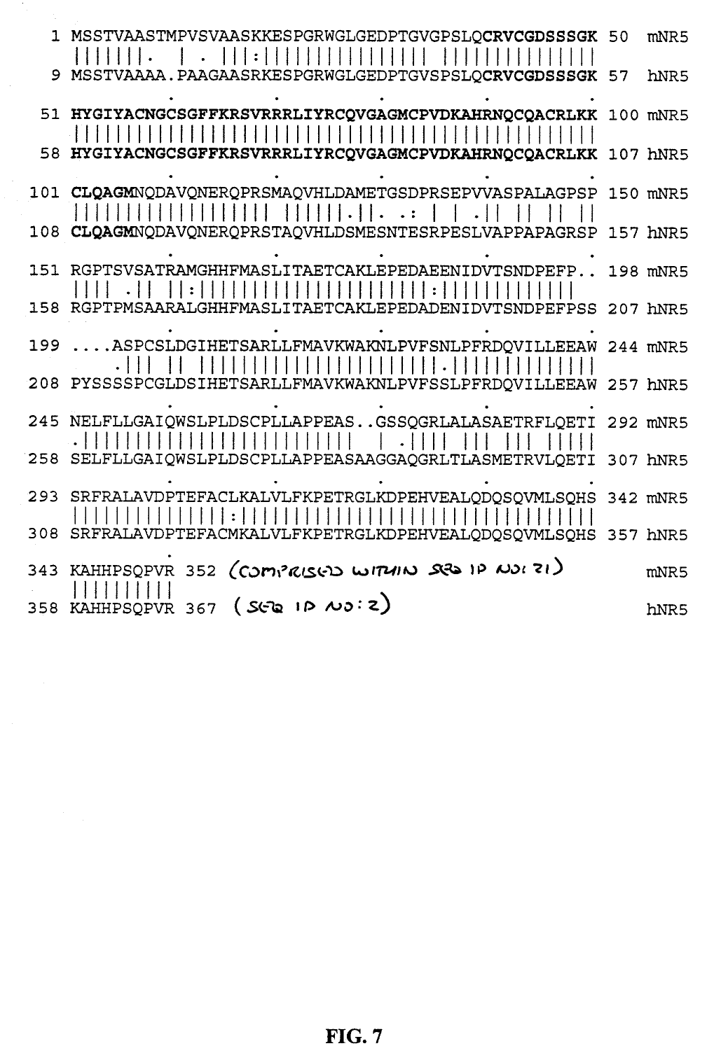

The oligonucleotide 5′-CCCAGGCTTTACACTTTATGCTTCC-3′ (SEQ ID NO:23) corresponding to pBluescript IISK vector was paired with primer hNR5R (5′-TGCCGCGTGTGCGGAGACAG-3′ [SEQ ID NO:24]) to amplify mNR5 5′ cDNA ends under degenerate PCR conditions. The template used was a λZap phage solution from a mouse eye cDNA library. DNA product from one round of PCR reaction was gel purified using a Qiagen gel extraction kit (Chatsworth, Calif.). The DNA fragments were then subcloned into PCRII™ TA vector (Invitrogen, Carlsbad, Calif.). Plasmid DNA from multiple clones were prepared and submitted for sequencing using the ABI PRISM™ dye terminator cycle sequencing ready reaction kit with AmpliTaq DNA polymerase, FS (Perkin Elmer, Norwalk, Conn.). The sequencing primers used were: TAF: 5′-GTACCGAGCTCGGATCCACTA-3′ (SEQ ID NO:25), TAR: 5′-CCGCCAGTGTGATGGATATCT-3′ (SEQ ID NO:26). Sequence assembly and analysis were performed with SEQUENCHER™ 3.0 (Gene Codes Corporation, Ann Arbor, Mich.). Ambiguities and/or discrepancies between automated base calling in sequencing reads were visually examined and edited to the correct base call. DNA sequences obtained showed about 80% DNA sequence identity with human nNR5. This sequence information was used to design an additional primer, mR5F1: 5′-TCGGTTGGGCCCAGCAACTTC-3′ (SEQ ID NO:27). MR5F1 was then paired with 5′-GGGGATGTGCTGCAAGGCGA-3′ (SEQ ID NO:28) from the vector and used to amplify the DNA sequence which comprises the entire open reading for mouse nNR5. FIGS. 4A-B shows the nucleotide sequence (SEQ ID NO:20) which comprises the open reading frame encoding the mouse nuclear receptor protein, nNR5. The coding, anticoding and amino acid sequence of mouse nNR5 are shown in FIGS. 5A-C. FIG. 6 also shows the amino acid sequence (SEQ ID NO: 21) of mouse nNR5, with the DNA binding domain from Cys-39 to Met-106. FIG. 7 shows the polypeptide alignment between mouse (comprised within SEQ ID NO:21) and human (comprised within SEQ ID NO:2) nuclear receptor 5 (nNR5). The overlapping region has 90% amino acid sequence identity. The DBD (DNA binding domain) are in bold, from Cys-39 to Met-106 of SEQ ID NO:21 and from Cys-47 to Met-13 of SEQ ID NO:2 (NR5).

### EXAMPLE 3

### Northern Analysis, In Situ Hybridization and Cell Based Assays of Human and Mouse nNR5

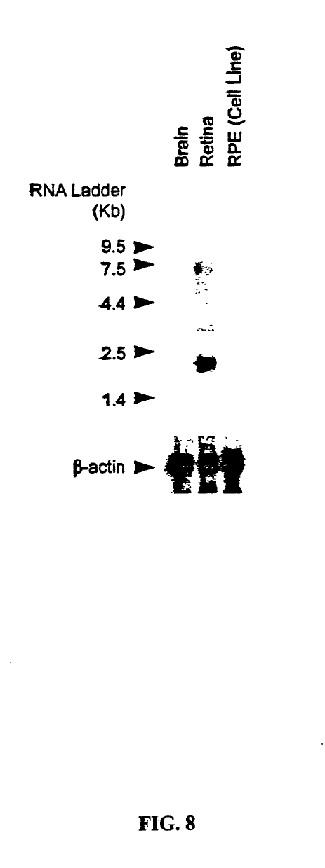

Northern Analysis—Northern analysis was performed on human brain, retina and human RPE (retina pigment epithelium) cells using a DNA probe corresponding to the entire ligand binding domain (LBD) of human nNR5. Three mRNA bands were detected in the human retina sample, while the nNR5 probe did not hybridize to the brain or PRE cells (FIG. 8). Each band corresponds to a unique splice variant. The ˜2.0 Kb band is the major transcript which encodes a protein comprising 410 amino acids. The ˜7.0 Kb band represents an mRNA that encodes the 367 amino acid protein described herein as SEQ ID NO:2. β-actin detection showed that all the lanes had equal amount of mRNA.

In Situ Hybridization For Mouse and Monkey Retinal Specific Nuclear Receptor (nNR5)—The plasmid PCRII (Invitrogen) containing a 383 base pair cDNA fragment coding for human nNR5 was linearized with either HindIII (Boehringer) or EcoR I (Boehringer) to generate template DNA for sense and antisense probes respectively. Template DNA for mouse specific RNR probes was similarly prepared using the plasmid PCRII containing a 380 bp mouse specific cDNA fragment linearized with SacI and EcoRV (Boehringer Manheim). Biotinylated sense and antisense riboprobes specific for mouse NR5 and human NR5 were generated via in vitro transcription using Biotin RNA Labeling Mix (Boehringer Mannheim). Whole eyes from eight-week-old Swiss Webster albino Mice were fixed by intracardiac perfusion with 4% paraformaldehyde processed embedded in paraffin and sectioned (8 μm). In situ hybridization was carried out as described (Petrukhin et al, 1998, Nature Genet. 19(3): 241-247) using the TSA Indirect Kit (NEN Life Sciences). Amplified signal was visualized with DAB (Sigma) solubilized in nickel chloride buffer (Digene). Whole monkey eyes were enucleated and fixed by immersion in 4% paraformaldehyde for 8 hrs. Following radial cuts and lens removal the eyes were cryoprotected in graded sucrose solutions and a sucrose/Tissue Tek O.C.T. Compound (Miles) mixture and frozen on dry ice as described (Barther et al, 1990, J. Histochem. Cytochem. 38:1383-1388). Retinal segments were sectioned (6 μm), and in situ hybridization was carried out as above using TSA Direct Red Fish according to manufacturer's instructions. Sections were counter-stained with 4,6-diamidino-2-phenylindole (DAPI, Molecular Probes) and images were digitally acquired using a Spot Cam CCD camera (Phase 3 Imaging Systems, Milford, Mass.).

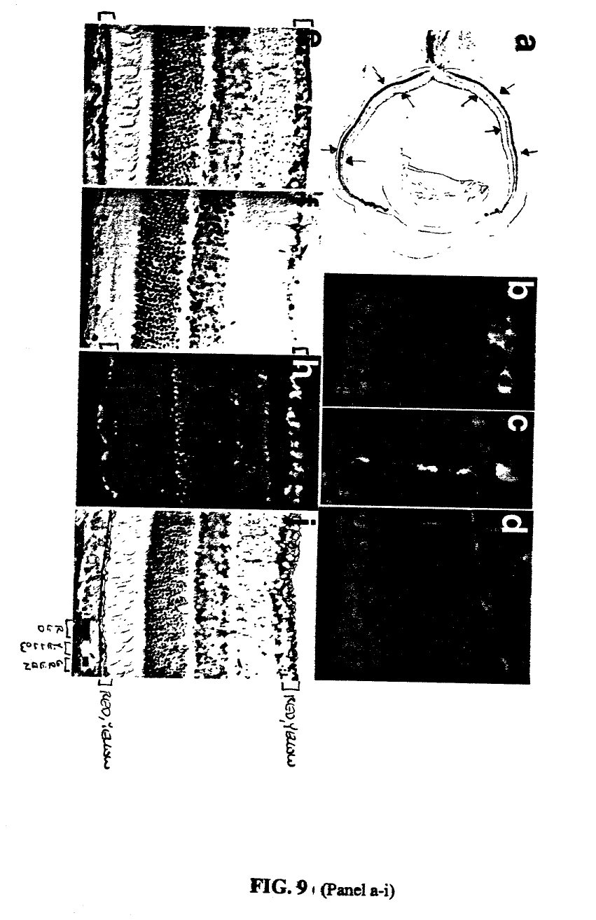

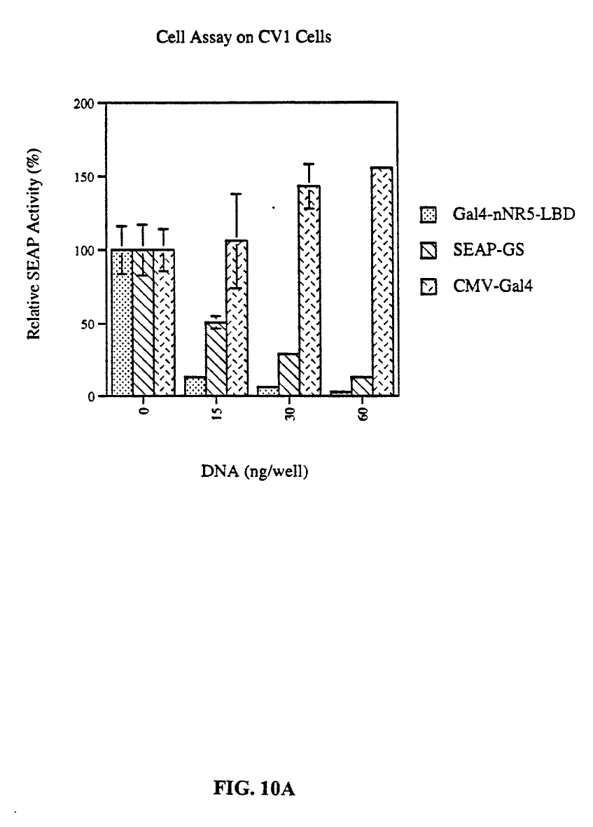

Mouse nNR5 probes in the LBD region and 3′-UTR generated the same result. The probe used for rhesus monkey tissue is specific for human nNR5, a splicing variant of hPNR as described by Kobayashi et al. (Proc. Natl. Acad. Sci 96: 4814-4819). These additional results are shown in FIG. 9, Panels a-i. Panel a is the whole mouse eye section. The blue staining (arrows in black and white drawing) indicates Muller Glial cells (inner layer, arrows in black and white drawing) and RPE cells (the thin outer layer, arrows in black and white drawing). Panel b, c and d are immunohistochemistry staining using Muller Glial cell specific markers—S100 and GFAP. The signal in red (light shade in black and white drawing) indicates Muller cells and its processes. Panel e is an amplification section of mouse retina. The black stain (bracketed region in black and white drawing) show expression of mouse nNR5 in Muller layler (top) and RPE layer (bottom). The dotted signals were nNR5 expression in Muller cell processes. Panel f is a mouse retina section hybridized with sense probe which is a negative control. No signal was detected in negative control (as indicated by a lack of bracketing in Panel f). Panel h and i are rhesus monkey retina sections. The same result is presented in two different image systems. In panel h, the blue fluoresent (light shaded portion of bracketed region in black and white drawing) shows the expression of nNR5, while in panel i, the expression level is in pseudo color. Yellow to green color indicated higher level of mRNA expression. The bracketed region of Panel h in the black and white drawing shows expression levels corresponding to red and yellow regions. The human result confirmed the mouse data showing nNR5 expression in Muller Glial and RPE cells.

Receptor and reporter co-transfection assays—A Ga14-nNR5-LBD construct and a SEAP reporter with Ga14 binding sites were cotransfected into either CV1 or RPE cells by techniques well known in the art. FIG. 10A shows that an increased amount of Ga14-nNR5-LBD reduces the SEAP expression level. This reduction is contributed through inhibiting promoter activity and squelching of transcription factors because partial inhibition is also observed on the SEAP reporter without Ga14 binding sites (SEAP-GS). A CMV vector containing Ga14 domain did not contribute to the inhibition. The same results were obtained with RPE-J cells transfected with the above-described constructs (FIG. 10B). FIG. 10C demonstrates that the inhibition function of nNR5-LBD is not observed on other nuclear receptors, such as, Erb-LBD or hPXR-LBD.

What is claimed:

1. A purified DNA molecule encoding a human nNR5 protein wherein said protein comprises the amino acid sequence as follows:
METRPTALMS STVAAAAPAA GAASRKESPG RWGLGEDPTG
VSPSLQCRVC
GDSSSGKHYG IYACNGCSGF FKRSVRRRLI YRCQVGAGMC
PVDKAHRNQC
QACRLKKCLQ AGMNQDAVQN ERQPRSTAQV HLDSMESNTE
SRPESLVAPP
APAGRSPRGP TPMSAARALG HHFMASLITA ETCAKLEPED
ADENIDVTSN
DPEFPSSPYS SSSPCGLDSI HETSARLLFM AVKWAKNLPV
FSSLPFRDQV
ILLEEAWSEL FLLGAIQWSL PLDSCPLLAP PEASAAGGAQ
GRLTLASMET
RVLQETISRF RALAVDPTEF ACMKALVLFK PETRGLKDPE
HVEALQDQSQ
VMLSQHSKAH HPSQPVR, as set forth in three-
letter abbreviation in SEQ ID NO:2.

2. An expression vector for expressing a human nNR5 protein in a recombinant host cell wherein said expression vector comprises a DNA molecule of claim 1.

3. A host cell which expresses a recombinant human nNR5 protein wherein said host cell contains the expression vector of claim 2.

4. A process for expressing a human nNR5 protein in a recombinant host cell, comprising:
(a) transfecting the expression vector of claim 2 into a suitable host cell; and,
(b) culturing the host cells of step (a) under conditions which allow expression of said the human nNR5 protein from said expression vector.

5. A purified DNA molecule encoding a human nNR5 protein wherein said protein consists of the amino acid sequence as follows:
METRPTALMS STVAAAAPAA GAASRKESPG RWGLGEDPTG
VSPSLQCRVC
GDSSSGKHYG IYACNGCSGF FKRSVRRRLI YRCQVGAGMC
PVDKAHRNQC
QACRLKKCLQ AGMNQDAVQN ERQPRSTAQV HLDSMESNTE
SRPESLVAPP
APAGRSPRGP TPMSAARALG HHFMASLITA ETCAKLEPED
ADENIDVTSN
DPEFPSSPYS SSSPCGLDSI HETSARLLFM AVKWAKNLPV
FSSLPFRDQV
ILLEEAWSEL FLLGAIQWSL PLDSCPLLAP PEASAAGGAQ
GRLTLASMET
RVLQETISRF RALAVDPTEF ACMKALVLFK PETRGLKDPE
HVEALQDQSQ
VMLSQHSKAH HPSQPVR, as set forth in three-
letter abbreviation in SEQ ID NO:2.

6. An expression vector for expressing a human nNR5 protein in a recombinant host cell wherein said expression vector comprises a DNA molecule of claim 5.

7. A host cell which expresses a recombinant human nNR5 protein wherein said host cell contains the expression vector of claim 6.

8. A process for expressing a human nNR5 protein in a recombinant host cell, comprising:
(a) transfecting the expression vector of claim 6 into a suitable host cell; and,
(b) culturing the host cells of step (a) under conditions which allow expression of said the human nNR5 protein from said expression vector.

9. A purified DNA molecule encoding a human nNR5 protein wherein said DNA molecule comprises the nucleotide sequence as set forth in SEQ ID NO: 1, as follows:
(SEQ ID NO:1)
ATTCGGGACC TTGGGGCAGC TCCTGAGTTC AGACAGAGTT
CAGGAAGGGA
GACAGGGGCA CAGAGAGACA GAGGTTCATG GACTGAGGCA
AAGGCTGGGC
CAGGCTCAGC AACCCAGGCC TCCCGCAGGC AGGCAGAGGC
TGCCCTGTAA
CCCATGGAGA CCAGACCAAC AGCTCTGATG AGCTCCACAG
TGGCTGCAGC
TGCGCCTGCA GCTGGGGCTG CCTCCAGGAA GGAGTCTCCA
GGCAGATGGG
GCCTGGGGGA GGATCCCACA GGCGTGAGCC CCTCGCTCCA
GTGCCGCGTG
TGCGGAGACA GCAGCAGCGG GAAGCACTAT GGCATCTATG
CCTGCAACGG
CTGCAGCGGC TTCTTCAAGA GGAGCGTACG GCGGAGGCTC
ATCTACAGGT
GCCAGGTGGG GGCAGGGATG TGCCCCGTGG ACAAGGCCCA
CCGCAACCAG
TGCCAGGCCT GCCGGCTGAA GAAGTGCCTG CAGGCGGGGA
TGAACCAGGA
CGCCGTGCAG AACGAGCGCC AGCCGCGAAG CACAGCCCAG
GTCCACCTGG
ACAGCATGGA GTCCAACACT GAGTCCCGGC CGGAGTCCCT
GGTGGCTCCC
CCGGCCCCGG CAGGGCGCAG CCCACGGGGC CCCACACCCA
TGTCTGCAGC
CAGAGCCCTG GGCCACCACT TCATGGCCAG CCTTATAACA
GCTGAAACCT
GTGCTAAGCT GGAGCCAGAG GATGCTGATG AGAATATTGA
TGTCACCAGC
AATGACCCTG AGTTCCCCTC CTCTCCATAC TCCTCTTCCT
CCCCCTGCGG
CCTGGACAGC ATCCATGAGA CCTCGGCTCG CCTACTCTTC
ATGGCCGTCA
AGTGGGCCAA GAACCTGCCT GTGTTCTCCA GCCTGCCCTT
CCGGGATCAG
GTGATCCTGC TGGAAGAGGC GTGGAGTGAA CTCTTTCTCC
TCGGGGCCAT
CCAGTGGTCT CTGCCTCTGG ACAGCTGTCC TCTGCTGGCA
CCGCCCGAGG
CTTCTGCTGC CGGTGGTGCC CAGGGCCGGC TCACGCTGGC
CAGCATGGAG
ACGCGTGTCC TGCAGGAAAC TATCTCTCGG TTCCGGGCAT
TGGCGGTGGA
CCCCACGGAG TTTGCCTGCA TGAAGGCCTT GGTCCTCTTC
AAGCCAGAGA
CGCGGGGCCT GAAGGATCCT GAGCACGTAG AGGCCTTGCA
GGACCAGTCC
CAAGTGATGC TGAGCCAGCA CAGCAAGGCC CACCACCCCA
GCCAGCCCGT
GAGGTGACCT GAGCATGCGC CCACCCACTC ATCTGTCCCT
GACCTCTAAC
CTTTCTCTGC CTCTCCCACA CTCTCCCAGA GCTCACTGAT
TAGACAGCAC
AAGGGTCTCA GTTCAACAGC ATACAGCCAA CATCTATGGT
GTCCCAGGCA
CAGTGCCAGG CCCCGGGAGT GGGGACCAAG ATGTACATAA
GACAAAGCTA
CTGCCTTCTA GAGACAACCG GCAGTGACCT CACTGAAGAC
AAAAACTGCC
CTAGCCAGGT ACTGAGGGTT GCATGAATCT GCAGGAGACA
GAGATCCCCT
TGCATGGGAA ACATAAAGCA GAATTGGGAG GGACTTTGTG
GAGACAGGGC
TGGACTTGAA AGGAAGAAGA AGTCTAAAAG AAAACATCAT
TTGCAAAGGG
AGAGAGGGGC AAGCATGATA TGTTGTTAGA ACAGGAGCCC
ACTTTGAAGG
TATAACAGGT TCCTGCCAGT GAGAAATGGG GAGAATAAGC
CAGAAAAGTA
CCCTAGGACC AGCCCGTTCA GGACTTTGAA TGCCAGCCAA
AGGCCACGTC
TGACTTGGGA GGCAGAGGGC AGCTACTGCA GGTTTCCGAG
CAGAGGGTCA
TACACAGGGC TGGACCTCAC GCAGACTGGC ATGGCCATGG
GTCCAGAGGA
TACTACTGGG AAGGGGATGG CAGCTACTGC CACCTTCCAG
ATGGTTCCAT
GGAGTTCTGA TCTTTGGGCA TGGCCAGGGG AAGCAGAAGG
GAGACTCTAG
GAGTTGAAAT GGGTCAGACC CGGTGTTTGG GTGAAGGTAA
GGAATGAGGG
AAGAGGAGCT CTTTG.

10. A DNA molecule of claim 9 which consists of nucleotide 154 to about nucleotide 1257 of SEQ ID NO: 1.

11. An expression vector for expressing a human nNR5 protein wherein said expression vector comprises a DNA molecule of claim 9.

12. An expression vector for expressing a human nNR5 protein wherein said expression vector comprises a DNA molecule of claim 10.

13. A host cell which expresses a recombinant human nNR5 protein wherein said host cell contains the expression vector of claim 11.

14. A host cell which expresses a recombinant human nNR5 protein wherein said host cell contains the expression vector of claim 12.

15. A process for expressing a human nNR5 protein in a recombinant host cell, comprising:
(a) transfecting the expression vector of claim 11 into a suitable host cell; and,
(b) culturing the host cells of step (a) under conditions which allow expression of said the human nNR5 protein from said expression vector.

16. A purified DNA molecule encoding a human nNR5 protein wherein said DNA molecule consists of the nucleotide sequence as set forth in SEQ ID NO: 1, as follows:
(SEQ ID NO: 1)
ATTCGGGACC TTGGGGCAGC TCCTGAGTTC AGACAGAGTT
CAGGAAGGGA GACAGGGGCA CAGAGAGACA GAGGTTCATG
GACTGAGGCA AAGGCTGGGC CAGGCTCAGC AACCCAGGCC
TCCCGCAGGC AGGCAGAGGC TGCCCTGTAA CCCATGGAGA
CCAGACCAAC AGCTCTGATG AGCTCCACAG TGGCTGCAGC
TGCGCCTGCA GCTGGGGCTG CCTCCAGGAA GGAGTCTCCA
GGCAGATGGG GCCTGGGGGA GGATCCCACA GGCCTGAGCC
CCTCGCTCCA GTGCCGCGTG TGCGGAGACA GCAGCAGCGG
GAAGCACTAT GGCATCTATG CCTGCAACGG CTGCAGCGGC
TTCTTCAAGA GGAGCGTACG GCGGAGGCTC ATCTACAGGT
GCCAGGTGGG GGCAGGGATG TGCCCCGTGG ACAAGGCCCA
CCGCAACCAG TGCCAGGCCT GCCGGCTGAA GAAGTGCCTG
CAGGCGGGGA TGAACCAGGA CGCCGTGCAG AACGAGCGCC
AGCCGCGAAG CACAGCCCAG GTCCACCTGG ACAGCATGGA
GTCCAACACT GAGTCCCGGC CGGAGTCCCT GGTGGCTCCC
CCGGCCCCGG CAGGGCGCAG CCCACGGGGC CCCACACCCA
TGTCTGCAGC CAGAGCCCTG GGCCACCACT TCATGGCCAG
CCTTATAACA GCTGAAACCT GTGCTAAGCT GGAGCCAGAG
GATGCTGATG AGAATATTGA TGTCACCAGC AATGACCCTG
AGTTCCCCTC CTCTCCATAC TCCTCTTCCT CCCCCTGCGG
CCTGGACAGC ATCCATGAGA CCTCGGCTCG CCTACTCTTC
ATGGCCGTCA AGTGGGCCAA GAACCTGCCT GTGTTCTCCA
GCCTGCCCTT CCGGGATCAG GTGATCCTGC TGGAAGAGGC
GTGGAGTGAA CTCTTTCTCC TCGGGGCCAT CCAGTGGTCT
CTGCCTCTGG ACAGCTGTCC TCTGCTGGCA CCGCCCGAGG
CTTCTGCTGC CGGTGGTGCC CAGGGCCGGC TCACGCTGGC
CAGCATGGAG ACGCGTGTCC TGCAGGAAAC TATCTCTCGG
TTCCGGGCAT TGGCGGTGGA CCCCACGGAG TTTGCCTGCA
TGAAGGCCTT GGTCCTCTTC AAGCCAGAGA CGCGGGGCCT
GAAGGATCCT GAGCACGTAG AGGCCTTGCA GGACCAGTCC
CAAGTGATGC TGAGCCAGCA CAGCAAGGCC CACCACCCCA
GCCAGCCCGT GAGGTGACCT GAGCATGCGC CCACCCACTC
ATCTGTCCCT GACCTCTAAC CTTTCTCTGC CTCTCCCACA
CTCTCCCAGA GCTCACTGAT TAGACAGCAC AAGGGTCTCA
GTTCAACAGC ATACAGCCAA CATCTATGGT GTCCCAGGCA
CAGTGCCAGG CCCCGGGAGT GGGGACCAAG ATGTACATAA
GACAAAGCTA CTGCCTTCTA GAGACAACCG GCAGTGACCT
CACTGAAGAC AAAAACTGCC CTAGCCAGGT ACTGAGGGTT
GCATGAATCT GCAGGAGACA GAGATCCCCT TGCATGGGAA
ACATAAAGCA GAATTGGGAG GGACTTTGTG GAGACAGGGC
TGGACTTGAA AGGAAGAAGA AGTCTAAAAG AAAACATCAT
TTGCAAAGGG AGAGAGGGGC AAGCATGATA TGTTGTTAGA
ACAGGAGCCC ACTTTGAAGG TATAACAGGT TCCTGCCAGT
GAGAAATGGG GAGAATAAGC CAGAAAAGTA CCCTAGGACC
AGCCCGTTCA GGACTTTGAA TGCCAGCCAA AGGCCACGTC
TGACTTGGGA GGCAGAGGGC AGCTACTGCA GGTTTCCGAG
CAGAGGGTCA TACACAGGGC TGGACCTCAC GCAGACTGGC
ATGGCCATGG GTCCAGAGGA TACTACTGGG AAGGGGATGG
CAGCTACTGC CACCTTCCAG ATGGTTCCAT GGAGTTCTGA
TCTTTGGGCA TGGCCAGGGG AAGCAGAAGG GAGACTCTAG
GAGTTGAAAT GGGTCAGACC CGGTGTTTGG GTGAAGGTAA
GGAATGAGGG AAGAGGAGCT CTTTG.

17. A DNA molecule of claim 16 which consists of nucleotide 154 to about nucleotide 1257 of SEQ ID NO: 1.

18. An expression vector for expressing a human nNR5 protein wherein said expression vector comprises a DNA molecule of claim 16.

19. An expression vector for expressing a human nNR5 protein wherein said expression vector comprises a DNA molecule of claim 17.

20. A host cell which expresses a recombinant human nNR5 protein wherein said host cell contains the expression vector of claim 18.

21. A host cell which expresses a recombinant human nNR5 protein wherein said host cell contains the expression vector of claim 19.

22. A process for expressing a human nNR5 protein in a recombinant host cell, comprising:
(a) transfecting the expression vector of claim 18 into a suitable host cell; and,
(b) culturing the host cells of step (a) under conditions which allow expression of said the human nNR5 protein from said expression vector.

23. A purified DNA molecule encoding a human nNR5 protein wherein said DNA molecule comprises the nucleotide sequence as set forth in SEQ ID NO: 19, as follows:

| | |
|---|---|
| TATAGGGCGA ATTGGGTACC GGGCCCCCCC TCGAGGTCGA CGGTATCGAT | (SEQ ID NO:19) |
| AAGCTTGATA TCGAATTCGA ATTCGGGACC TTGGGGCAGC TCCTGAGTTC | |
| AGACAGAGTT CAGGAAGGGA GACAGGGGCA CAGAGAGACA GAGGTTCATG | |
| GACTGAGGCA AAGGCTGGGC CAGGCTCAGC AACCCAGGCC TCCCGCAGGC | |
| AGGCAGAGGC TGCCCTGTAA CCCATGGAGA CCAGACCAAC AGCTCTGATG | |
| AGCTCCACAG TGGCTGCAGC TGCGCCTGCA GCTGGGGCTG CCTCCAGGAA | |
| GGAGTCTCCA GGCAGATGGG GCCTGGGGGA GGATCCCACA GGCGTGAGCC | |
| CCTCGCTCCA GTGCCGCGTG TGCGGAGACA GCAGCAGCGG GAAGCACTAT | |
| GGCATCTATG CCTGCAACGG CTGCAGCGGC TTCTTCAAGA GGAGCGTACG | |
| GCGGAGGCTC ATCTACAGGT GCCAGGTGGG GGCAGGGATG TGCCCCGTGG | |
| ACAAGGCCCA CCGCAACCAG TGCCAGGCCT GCCGGCTGAA GAAGTGCCTG | |
| CAGGCGGGGA TGAACCAGGA CGCCGTGCAG AACGAGCGCC AGCCGCGAAG | |
| CACAGCCCAG GTCCACCTGG ACAGCATGGA GTCCAACACT GAGTCCCGGC | |
| CGGAGTCCCT GGTGGCTCCC CCGGCCCCGG CAGGGCGCAG CCCACGGGGC | |
| CCCACACCCA TGTCTGCAGC CAGAGCCCTG GGCCACCACT TCATGGCCAG | |
| CCTTATAACA GCTGAAACCT GTGCTAAGCT GGAGCCAGAG GATGCTGATG | |
| AGAATATTGA TGTCACCAGC AATGACCCTG AGTTCCCCTC CTCTCCATAC | |
| TCCTCTTCCT CCCCCTGCGG CCTGGACAGC ATCCATGAGA CCTCGGCTCG | |
| CCTACTCTTC ATGGCCGTCA AGTGGGCCAA GAACCTGCCT GTGTTCTCCA | |
| GCCTGCCCTT CCGGGATCAG GTGATCCTGC TGGAAGAGGC GTGGAGTGAA | |
| CTCTTTCTCC TCGGGGCCAT CCAGTGGTCT CTGCCTCTGG ACAGCTGTCC | |
| TCTGCTGGCA CCGCCCGAGG CCTCTGCTGC CGGTGGTGCC CAGGGCCGGC | |
| TCACGCTGGC CAGCATGGAG ACGCGTGTCC TGCAGGAAAC TATCTCTCGG | |
| TTCCGGGCAT TGGCGGTGGA CCCCACGGAG TTTGCCTGCA TGAAGGCCTT | |
| GGTCCTCTTC AAGCCAGAGA CGCGGGGCCT GAAGGATCCT GAGCACGTAG | |
| AGGCCTTGCA GGACCAGTCC CAAGTGATGC TGAGCCAGCA CAGCAAGGCC | |
| CACCACCCCA GCCAGCCCGT GAGGTGACCT GAGCATGCGC CCACCCACTC | |
| ATCTGTCCCT GACCTCTAAC CTTTCTCTGC CTCTCCCACA CTCTCCCAGA | |
| GCTCACTGAT TAGACAGCAC AAGGGTCTCA GTTCAACAGC ATACAGCCAA | |
| CATCTATGGT GTCCCAGGCA CAGTGCCAGG CCCCGGGAGT GGGGACCAAG | |
| ATGTACATAA GACAAAGCTA CTGCCTTCTA GAGACAACCG GCAGTGACCT | |
| CACTGAAGAC AAAAACTGCC CTAGCCAGGT ACTGAGGGTT GCATGAATCT | |
| GCAGGAGACA GAGATCCCCT TGCATGGGAA ACATAAAGCA GAATTGGGAG | |
| GGACTTTGTG GAGACAGGGC TGGACTTGAA AGGAAGAAGA AGTCTAAAAG | |
| AAAACATCAT TTGCAAAGGG AGAGAGGGGC AAGCATGATA TGTTGTTAGA | |
| ACAGGAGCCC ACTTTGAAGG TATAACAGGT TCCTGCCAGT GAGAAATGGG | |
| GAGAATAAGC CAGAAAAGTA CCCTAGGACC AGCCCGTTCA GGACTTTGAA | |
| TGCCAGCCAA AGGCCACGTC TGACTTGGGA GGCAGAGGGC AGCTACTGCA | |
| GCTTTCCGAG CAGAGGGTCA TACACAGGGC TGGACCTCAC GCAGACTGGC | |
| ATGGCCATGG GTCCAGAGGA TACTACTGGG AAGGGGATGG CAGCTACTGC | |
| CACCTTCCAG ATGGTTCCAT GGAGTTCTGA TCTTTGGGCA TGGCCAGGGG | |
| AAGCAGAAGG GAGACTCTAG GAGTTGAAAT GGGTCAGACC CGGTGTTTGG | |
| GTGAAGGTAA GGAATGAGGG AAGAGGAGCT CTTTG. | |

24. An expression vector for expressing a human nNR5 protein wherein said expression vector comprises a DNA molecule of claim 23.

25. A host cell which expresses a recombinant human nNR5 protein wherein said host cell contains the expression vector of claim 24.

26. A process for expressing a human nNR5 protein in a recombinant host cell, comprising:
(a) transfecting the expression vector of claim 24 into a suitable host cell; and,
(b) culturing the host cells of step (a) under conditions which allow expression of said the human nNR5 protein from said expression vector.

27. A DNA molecule of claim 23 which consists of nucleotide 224 to about nucleotide 1327 of SEQ ID NO: 19.

28. A purified human nNR5 protein which comprises the amino acid sequence as set forth in SEQ ID NO: 2.

29. The purified human nNR5 protein of claim 28 which consists of the amino acid sequence as set forth in SEQ ID NO: 2.

30. A purified DNA molecule encoding a mouse nNR5 protein wherein said protein comprises the amino acid sequence as follows:
MSSTVAASTM PVSVAASKKE SPGRWGLGED PTGVGPSLQC
RVCGDSSSGK HYGIYACNGC SGFFKRSVRR RLIYRCQVGA
GMCPVDKAHR NQCQACRLKK CLQAGMNQDA VQNERQPRSM
AQVHLDAMET GSDPRSEPVV ASPALAGPSP RGPTSVSATR
AMGHHFMASL ITAETCAKLE PEDAEENIDV TSNDPEFPAS
PCSLDGIHET SARLLFMAVK WAKNLPVFSN LPFRDQVILL
EEAWNELFLL GAIQWSLPLD SCPLLAPPEA SGSSQGRLAL
ASAETRFLQE TISRFRALAV DPTEFACLKA LVLFKPETRG
LKDPEHVEAL QDQSQVMLSQ HSKAHHPSQP VRFGKLLLLL
PSLRFLTAER IELLFFRKTI GNTPMEKLLC DMFKN, as set
forth in the three-letter abbreviation in
SEQ ID NO:21.

31. An expression vector for expressing a mouse nNR5 protein in a recombinant host cell wherein said expression vector comprises a DNA molecule of claim 30.

32. A host cell which expresses a recombinant mouse nNR5 protein wherein said host cell contains the expression vector of claim 31.

33. A process for expressing a mouse nNR5 protein in a recombinant host cell, comprising:
(a) transfecting the expression vector of claim 31 into a suitable host cell; and,
(b) culturing the host cells of step (a) under conditions which allow expression of said the mouse nNR5 protein from said expression vector.

34. A purified DNA molecule encoding a mouse nNR5 protein wherein said protein consists of the amino acid sequence as follows:
MSSTVAASTM PVSVAASKKE SPGRWGLGED PTGVGPSLQC
RVCGDSSSGK HYGIYACNGC SGFFKRSVRR RLIYRCQVGA
GMCPVDKAHR NQCQACRLKK CLQAGMNQDA VQNERQPRSM
AQVHLDAMET GSDPRSEPVV ASPALAGPSP RGPTSVSATR
AMGHHFMASL ITAETCAKLE PEDAEENIDV TSNDPEFPAS
PCSLDGIHET SARLLFMAVK WAKNLPVFSN LPFRDQVILL
EEAWNELFLL GAIQWSLPLD SCPLLAPPEA SGSSQGRLAL
ASAETRFLQE TISRFRALAV DPTEFACLKA LVLFKPETRG
LKDPEHVEAL QDQSQVMLSQ HSKAHHPSQP VRFGKLLLLL
PSLRFLTAER IELLFFRKTI GNTPMEKLLC DMFKN, as set
forth in three-letter abbreviation in
SEQ ID NO:21.

35. An expression vector for expressing a mouse nNR5 protein in a recombinant host cell wherein said expression vector comprises a DNA molecule of claim 34.

36. A host cell which expresses a recombinant mouse nNR5 protein wherein said host cell contains the expression vector of claim 35.

37. A process for expressing a mouse nNR5 protein in a recombinant host cell, comprising:
(a) transfecting the expression vector of claim 35 into a suitable host cell; and,
(b) culturing the host cells of step (a) under conditions which allow expression of said the mouse nNR5 protein from said expression vector.

38. A purified DNA molecule encoding a mouse nNR5 protein wherein said DNA molecule comprises the nucleotide sequence as set forth in SEQ ID NO: 20, as follows:

| | |
|---|---|
| TCGGTTGGGC CCAGCAACTT CTAGCAAGCA GGCTACCCTT AGGACCATCC | (SEQ ID NO:20) |
| ATATCCGATG AGCTCTACAG TGGCTGCCTC CACTATGCCT GTGTCTGTGG | |
| CGGCCTCCAA GAAGGAGTCT CCAGGTAGAT GGGGCCTTGG AGAGGATCCA | |
| ACAGGTGTGG GCCCCTCGCT CCAGTGCCGA GTGTGTGGGG ACAGCAGCAG | |
| TGGGAAACAT TATGGCATCT ATGCCTGCAA TGGCTGCAGT GGCTTCTTCA | |
| AGAGGAGTGT GAGAAGGAGG CTCATCTACA GGTGCCAAGT CGGGGCAGGG | |
| ATGTGCCCAG TGGATAAGGC CCATCGCAAT CAGTGCCAGG CCTGCCGGCT | |
| GAAGAAGTGC TTACAAGCAG GCATGAACCA AGATGCTGTG CAGAATGAGC | |
| GCCAACCTCG GAGCATGGCT CAGGTCCACC TGGATGCCAT GGAAACAGGC | |
| AGTGACCCCC GATCAGAACC AGTGGTAGCC TCTCCTGCTC TGGCAGGGCC | |
| CAGTCCCCGG GGCCCCACGT CTGTGTCTGC AACCAGAGCC ATGGGCCACC | |
| ACTTTATGGC CAGCCTTATC ACCGCCGAAA CTTGTGCTAA ACTGGAGCCA | |
| GAGGACGCTG AAGAGAATAT TGATGTCACC AGCAATGACC CCGAGTTCCC | |
| CGCATCCCCC TGCAGTCTGG ATGGCATCCA TGAGACATCT GCTCGCCTGC | |
| TCTTCATGGC TGTCAAATGG GCCAAAAACT TGCCTGTGTT TTCCAACCTG | |
| CCTTTCCGGG ACCAGGTGAT CTTGCTGGAA GAGGCATGGA ATGAGCTTTT | |
| CCTTCTTGGA GCCATACAGT GGTCTCTGCC CCTGGACAGC TGCCCACTGC | |
| TGGCACCACC TGAGGCGTCC GGCAGCTCTC AGGGCAGGCT GGCCTTGGCC | |
| AGTGCAGAGA CGCGCTTCCT GCAGGAAACC ATCTCCCGGT TCCGAGCTCT | |
| GGCAGTGGAT CCCACAGAGT TTGCCTGCCT GAAGGCCCTG GTCCTCTTCA | |
| AACCTGAAAC ACGAGGCCTG AAGGATCCTG AGCACGTGGA GGCTTTGCAG | |
| GACCAGTCCC AGGTGATGCT AAGCCAGCAT AGCAAGGCTC ACCACCCCAG | |
| CCAGCCTGTG AGGTTTGGGA AATTGCTCCT CCTGCTCCCA TCTTTGAGGT | |
| TCCTCACGGC TGAGCGCATT GAGCTTCTCT TCTTCAGAAA GACCATAGGO | |
| AACACTCCGA TGGAGAAGCT CCTGTGTGAC ATGTTCAAAA ACTAGTTGGG | |
| AGTGCCAAGT GTCCACAGGC ACCCAGGGGG GCAGCACATC TTAGAAGCTA | |
| AATAGTTCCC TGCCTTTCTC AGCCAGTAAT TCCACATTCA GGTATTCCTA | |
| CCTAGCAGAA ATTTCTCCCA AAATATATTA TTGGCATATT CATTGCCATC | |
| CTAATCTTAA TACCCCTAAC TCTGCTTGGG CAGTAGAATG CATGGATGCG | |
| TTGTTATATT CATAGGAGAA ACAGCTTTGG CAAAAAAAAA AAAAAAAAAA | |
| AAAAAAAAAA AAAAAAAAAA AAAAAAAAAA CTCGAGGGGG GGCCCGGTAC | |
| CCAATTCGCC CTATAGTGAG TCGTATTACA ATTCACTGGC CGTCGTTTTA | |
| CAACGTCGTG ACTGGGAAAA CCC. | |

39. A DNA molecule of claim 38 which consists of nucleotide 58 to about nucleotide 1245 of SEQ ID NO: 20.

40. An expression vector for expressing a mouse nNR5 protein wherein said expression vector comprises a DNA molecule of claim 38.

41. An expression vector for expressing a mouse nNR5 protein wherein said expression vector comprises a DNA molecule of claim 39.

42. A host cell which expresses a recombinant mouse nNR5 protein wherein said host cell contains the expression vector of claim 40.

43. A host cell which expresses a recombinant mouse nNR5 protein wherein said host cell contains the expression vector of claim 41.

44. A process for expressing a mouse nNR5 protein in a recombinant host cell, comprising:
(a) transfecting the expression vector of claim 40 into a suitable host cell; and,
(b) culturing the host cells of step (a) under conditions which allow expression of said the mouse nNR5 protein from said expression vector.

45. A purified DNA molecule encoding a mouse nNR5 protein wherein said DNA molecule consists of the nucleotide sequence as set forth in SEQ ID NO: 20, as follows:

| | |
|---|---|
| TCGGTTGGGC CCAGCAACTT CTAGCAAGCA GGCTACCCTT AGGACCATCC | (SEQ ID NO:20). |
| ATATCCGATG AGCTCTACAG TGGCTGCCTC CACTATGCCT GTGTCTGTGG | |
| CGGCCTCCAA GAAGGAGTCT CCAGGTAGAT GGGGCCTTGG AGAGGATCCA | |
| ACAGGTGTGG GCCCCTCGCT CCAGTGCCGA GTGTGTGGGG ACAGCAGCAG | |
| TGGGAAACAT TATGGCATCT ATGCCTGCAA TGGCTGCAGT GGCTTCTTCA | |
| AGAGGAGTGT GAGAAGGAGG CTCATCTACA GGTGCCAAGT CGGGGCAGGG | |
| ATGTGCCCAG TGGATAAGGC CCATCGCAAT CAGTGCCAGG CCTGCCGGCT | |
| GAAGAAGTGC TTACAAGCAG GCATGAACCA AGATGCTGTG CAGAATGAGC | |
| GCCAACCTCG GAGCATGGCT CAGGTCCACC TGGATGCCAT GGAAACAGGC | |
| AGTGACCCCC GATCAGAACC AGTGGTAGCC TCTCCTGCTC TGGCAGGGCC | |
| CAGTCCCCGG GGCCCCACGT CTGTGTCTGC AACCAGAGCC ATGGGCCACC | |
| ACTTTATGGC CAGCCTTATC ACCGCCGAAA CTTGTGCTAA ACTGGAGCCA | |
| GAGGACGCTG AAGAGAATAT TGATGTCACC AGCAATGACC CCGAGTTCCC | |
| CGCATCCCCC TGCAGTCTGG ATGGCATCCA TGAGACATCT GCTCGCCTGC | |
| TCTTCATGGC TGTCAAATGG GCCAAAAACT TGCCTGTGTT TTCCAACCTG | |
| CCTTTCCGGG ACCAGGTGAT CTTGCTGGAA GAGGCATGGA ATGAGCTTTT | |
| CCTTCTTGGA GCCATACAGT GGTCTCTGCC CCTGGACAGC TGCCCACTGC | |
| TGGCACCACC TGAGGCGTCC GGCAGCTCTC AGGGCAGGCT GGCCTTGGCC | |
| AGTGCAGAGA CGCGCTTCCT GCAGGAAACC ATCTCCCGGT TCCGAGCTCT | |
| GGCAGTGGAT CCCACAGAGT TTGCCTGCCT GAAGGCCCTG GTCCTCTTCA | |
| AACCTGAAAC ACGAGGCCTG AAGGATCCTG AGCACGTGGA GGCTTTGCAG | |
| GACCAGTCCC AGGTGATGCT AAGCCAGCAT AGCAAGGCTC ACCACCCCAG | |
| CCAGCCTGTG AGGTTTGGGA AATTGCTCCT CCTGCTCCCA TCTTTGAGGT | |
| TCCTCACGGC TGAGCGCATT GAGCTTCTCT TCTTCAGAAA GACCATAGGG | |
| AACACTCCGA TGGAGAAGCT CCTGTGTGAC ATGTTCAAAA ACTAGTTGGG | |
| AGTGCCAAGT GTCCACAGGC ACCCAGGGGG GCAGCACATC TTAGAAGCTA | |
| AATAGTTCCC TGCCTTTCTC AGCCAGTAAT TCCACATTCA GGTATTCCTA | |
| CCTAGCAGAA ATTTCTCCCA AAATATATTA TTGGCATATT CATTGCCATC | |
| CTAATCTTAA TACCCCTAAC TCTGCTTGGG CAGTAGAATG CATGGATGCG | |
| TTGTTATATT CATAGGAGAA ACAGCTTTGG CAAAAAAAAA AAAAAAAAAA | |
| CCAATTCGCC CTATAGTGAG TCGTATTACA ATTCACTGGC CGTCGTTTTA | |
| CAACGTCGTG ACTGGGAAAA CCC. | |

46. A DNA molecule of claim 45 which consists of nucleotide 58 to about nucleotide 1245 of SEQ ID NO: 20.

47. An expression vector for expressing a mouse nNR5 protein wherein said expression vector comprises a DNA molecule of claim 45.

48. An expression vector for expressing a mouse nNR5 protein wherein said expression vector comprises a DNA molecule of claim 46.

49. A host cell which expresses a recombinant mouse nNR5 protein wherein said host cell contains the expression vector of claim 47.

50. A host cell which expresses a recombinant mouse nNR5 protein wherein said host cell contains the expression vector of claim 48.

51. A process for expressing a mouse nNR5 protein in a recombinant host cell, comprising:
(a) transfecting the expression vector of claim 47 into a suitable host cell; and,
(b) culturing the host cells of step (a) under conditions which allow expression of said the mouse nNR5 protein from said expression vector.

52. A purified mouse nNR5 protein which comprises the amino acid sequence as set forth in SEQ ID NO: 21.

53. The purified mouse nNR5 protein of claim 52 which consists of the amino acid sequence as set forth in SEQ ID NO: 21.

54. A method of identifying modulators of nNR5 activity, comprising:
(a) combining a modulator of nNR5 activity with a nNR5 protein or biologically equivalent portion thereof; and,
(b) measuring the effect of a modulator on the nNR5 activity.

55. The method of claim 54 wherein said nNR5 protein comprises the amino acid as set forth in SEQ ID NO:2.

56. The method of claim 54 wherein said nNR5 protein comprises the amino acid as set forth in SEQ ID NO:21.

57. A DNA molecule which comprises the nucleotide sequence as set forth in SEQ ID NO:18.
